SEQUENCE LISTING (7 sequences)

SEQ ID NO: 1 - protein, 143 aa, Homo sapiens
MSGRGKTGGKARAKAKSRSSRAGLQFPVGRVHRLLRKGHYAERVGAGAPVYLAAVLEYLTAEILELAGNAARDNKKTRIIPRHLQLAIRNDEELNKLLGGVTIAQGGVLPNIQAVLLPKKTSATVGPKAPSGGKKATQASQEY

SEQ ID NO: 2 - protein, 128 aa, Artificial Sequence
  19D12/15H12 Light Chain-C (LCC)
MSPSQLIGFLLLWVPASRGEIVLTQSPDSLSVTPGERVTITCRASQSIGSSLHWYQQKPGQSPKLLIKYASQSLSGVPSRFSGSGSGTDFTLTISSLEAEDAAAYYCHQSSRLPHTFGQGTKVEIKRT

SEQ ID NO: 3 - protein, 128 aa, Artificial Sequence
  19D12/15H12 Light Chain-D (LCD)
MSPSQLIGFLLLWVPASRGEIVLTQSPDSLSVTPGERVTITCRASQSIGSSLHWYQQKPGQSPKLLIKYASQSLSGVPSRFSGSGSGTDFTLTISSLEAEDFAVYYCHQSSRLPHTFGQGTKVEIKRT

SEQ ID NO: 4 - protein, 128 aa, Artificial Sequence
  19D12/15H12 Light Chain-E (LCE)
MSPSQLIGFLLLWVPASRGEIVLTQSPGTLSVSPGERATLSCRASQSIGSSLHWYQQKPGQAPRLLIKYASQSLSGIPDRFSGSGSGTDFTLTISRLEPEDAAAYYCHQSSRLPHTFGQGTKVEIKRT

SEQ ID NO: 5 - protein, 128 aa, Artificial Sequence
  19D12/15H12 Light Chain-F (LCF)
MSPSQLIGFLLLWVPASRGEIVLTQSPGTLSVSPGERATLSCRASQSIGSSLHWYQQKPGQAPRLLIKYASQSLSGIPDRFSGSGSGTDFTLTISRLEPEDFAVYYCHQSSRLPHTFGQGTKVEIKRT

SEQ ID NO: 6 - protein, 137 aa, Artificial Sequence
  19D12/15H12 heavy chain-A (HCA)
MEFGLSWVFLVAILKGVQCEVQLVQSGGGLVKPGGSLRLSCAASGFTFSSFAMHWVRQAPGKGLEWISVIDTRGATYYADSVKGRFTISRDNAKNSLYLQMNSLRAEDTAVYYCARLGNFYYGMDVWGQGTTVTVSS

SEQ ID NO: 7 - protein, 137 aa, Artificial Sequence
  19D12/15H12 heavy chain-B (HCB)
MEFGLSWVFLVAILKGVQCEVQLVQSGGGLVQPGGSLRLSCAASGFTFSSFAMHWVRQAPGKGLEWISVIDTRGATYYADSVKGRFTISRDNAKNSLYLQMNSLRAEDTAVYYCARLGNFYYGMDVWGQGTTVTVSS

(19) United States
(12) Patent Application Publication
(10) Pub. No.: US 2011/0009387 A1
(43) Pub. Date: Jan. 13, 2011

(54) HISTONE H2AX (HH2AX) BIOMARKER FOR FTI SENSITIVITY

(76) Inventor: Andrea Dawn Basso-Porcaro, Guttenberg, NJ (US)

Correspondence Address: MERCK;PATENT DEPARTMENT (K-6-1, 1990), 2000 GALLOPING HILL ROAD, KENILWORTH, NJ, 07033-0530, US

(21) Appl. No.: 12/601,643

(22) PCT Filed: Jun. 11, 2008
(86) PCT No.: PCT/US2008/007294

§ 371 Date: Apr. 13, 2010

Related U.S. Application Data
(60) Provisional application No. 60/943,353, filed on Jun. 12, 2007.

(51) Int. Cl. A61K 31/55; G01N 33/574; A61K 31/435; A61K 31/4709; A61K 31/336; A61K 31/192; A61K 31/24; A61K 31/497; A61K 31/551; A61P 35/00

(52) U.S. Cl. 514/212.08; 435/7.23; 514/290; 514/312; 514/475; 514/568; 514/541; 514/253.03; 514/221

(57) ABSTRACT

The present invention relates e.g., to methods for predicting cellular sensitivity to farnesyl protein transferase inhibitors, such as lonafarnib; manumycin A; FTI-276; L-744832; BMS-214662; tipifarnib; BMS-316810K. The methods involve determining if malignant cells exhibit increased expression. of phosphorylated histone H2Ax following contact of one or more of said cells with said inhibitor.

This application claims the benefit of U.S. provisional patent application No. 60/943,353; filed Jun. 12, 2007; which is herein incorporated by reference in its entirety.

### FIELD OF THE INVENTION

The present invention relates, generally, to methods for predicting sensitivity of a tumor to an FTI.

### BACKGROUND OF THE INVENTION

Farnesyl protein transferase (FPT) inhibitors (FTIs) are a current area of interest in the treatment and prevention of cancerous conditions. Indeed, there are several FTIs currently in clinical development or on the market. Examples of such FTIs include lonafarnib (Sarasar™; Schering Corporation; Kenilworth, N.J.) and tipifarnib (Zarnestra®; Johnson & Johnson).

Selection of patients with tumors which are likely to be responsive to a given FTI is also of interest since it decreases the chances of the futile administration of the inhibitor to a non-responsive patient. Such futile administrations are wasteful of both money and time. When time is of the essence, e.g., in the case of an aggressive tumor, there is a particular interest in finding an effective treatment as soon as possible. Another benefit of such patient selection relates to patient compliance. Patients assured that a given FTI therapy will likely be effective against their specific tumor will exhibit an enhanced likelihood of continuing with the prescribed FTI regimen over time. Tumor-specific characteristics that are associated with responsiveness to an FTI, such as the expression of one or more specific genes, can be used as biomarkers for the likelihood of sensitivity to that FTI. Accordingly, patients suffering from tumors expressing any of such biomarkers can be selected for treatment with an FTI. This approach of patient selection has been employed successfully in connection with other cancer treatments. For example, Bunn et al. report selection criteria for patients with non-small cell lung cancer for treatment with an epidermal growth factor receptor (EGFR) tyrosine kinase inhibitor (Clin. Cancer Res. 12: 3652-3656 (2006)). Han et al. identified markers (EGFR mutation, K-ras Mutation and Akt Phosphorylation) pointing to a likelihood of sensitivity to gefitinib (Clin. Cancer Res. 12: 2538-2544 (2006)).

Currently, there is a need in the art for the identification of biomarkers indicating a likelihood of FTI sensitivity. The present invention provides a convenient biomarker for FTI sensitivity which may be used in the clinic or doctor's office for, e.g., patient selection or FTI sensitivity prediction.

### SUMMARY OF THE INVENTION

The present invention addresses this need and others for example by providing methods and compositions useful for identifying patients, with a disease or medical condition, e.g., cancer, that is sensitive or likely to be sensitive to a given FTI.

The present invention provides a method for evaluating sensitivity of malignant cells (e.g., which are in a tumor such as a breast tumor, an ovarian tumor, a pancreatic tumor, a prostate tumor, a colon tumor, a brain tumor, a urothelial tract tumor, a non-small cell lung tumor, and a taxane refractory/resistant non-small cell lung tumor or which mediate a cancerous condition such as chronic myelogenous leukemia) to a farnesyl protein transferase inhibitor (e.g., lonafarnib; manumycin A; FTI-276; L-744832; BMS-214662; tipifarnib; BMS-316810;

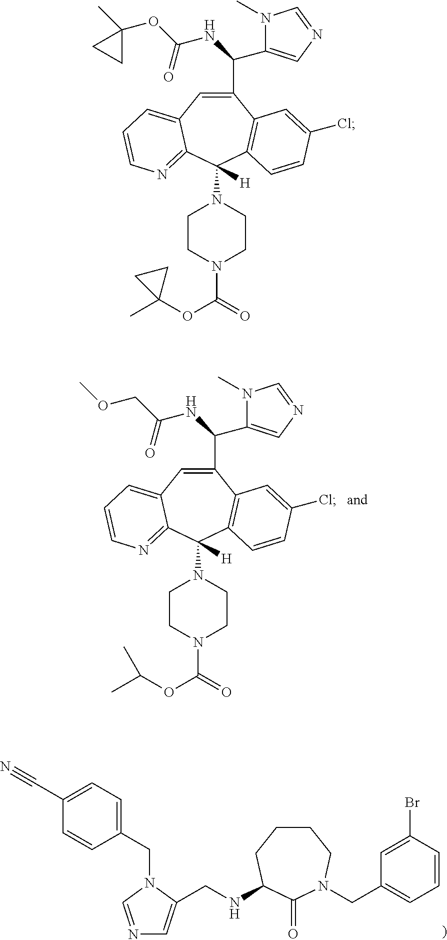

comprising determining if said cells exhibit increased expression of phosphorylated histone H2Ax following contact of one or more of said cells with said inhibitor wherein said cells are determined to be sensitive if said increased expression is observed. In an embodiment of the invention, the method further comprises administering a therapeutically effective dosage of said inhibitor, optionally in association with a further therapeutic agent (e.g., temozolomide, an isolated antibody that binds specifically to IGF1R, anastrazole, paclitaxel, docetaxel, taxane and gemcitabine), to the body of a subject comprising said malignant cells if the cells are determined to be sensitive. Said malignant cells may be obtained from an in vitro source (e.g., from a cell culture or ordered commercially, such as from the American Type Culture Collection (ATCC)) or may be obtained from an in vivo source (e.g., from a tumor in the body of a patient or from a blood sample from a patient with a non-solid cancer such a leukemia). Said phospho-histone H2Ax may be measured, for example, by western blot analysis. In an embodiment of the invention, the method comprises (a) obtaining a sample of one or more malignant cells from a subject not yet treated with said inhibitor; (b) evaluating expression of phosphorylated histone H2Ax in the malignant cell(s); (c) treating the subject with the inhibitor; (d) obtaining a second sample of one or more malignant cells from the subject treated with the inhibitor; (e) evaluating expression of the phosphorylated histone H2Ax in the cell(s) obtained from the subject treated with the inhibitor; wherein the cell is determined to be sensitive to the inhibitor if expression of the phosphorylated histone H2Ax is observed to increase following the treatment. In an embodiment of the invention, the subject is administered a therapeutically effective dose of said inhibitor, optionally in association with a further therapeutic agent (e.g., an anti-cancer agent such as temozolomide, an isolated antibody that binds specifically to IGF1R, anastrazole, paclitaxel, docetaxel, taxane and gemcitabine) if the cells are determined to be sensitive.

The present invention also provides a method for selecting a subject with malignant cells for treatment with a farnesyl protein transferase inhibitor comprising evaluating sensitivity of the malignant cells to said inhibitor, e.g., as set forth above; wherein said subject is selected if said cells are determined to be sensitive. In an embodiment of the invention, after the subject is selected, the subject is administered a therapeutically effective dosage of a farnesyl protein transferase inhibitor optionally in association with a further therapeutic agent.

The present invention also provides a method for identifying a subject with malignant cells sensitive or likely to be sensitive to a farnesyl protein transferase inhibitor comprising evaluating sensitivity of the malignant cells to said inhibitor; wherein said subject is identified if said cells are determined to be sensitive. In an embodiment of the invention, after the subject is identified, the subject is administered a therapeutically effective dosage of a farnesyl protein transferase inhibitor optionally in association with a further therapeutic agent.

The present invention also provides a method for treating a tumor or cancerous condition with a farnesyl protein transferase inhibitor comprising evaluating sensitivity of malignant cells, which are in said tumor or which mediate said cancerous condition, to said inhibitor and, if said cells are determined to be sensitive, continuing treatment by administering, to the subject, a therapeutically effective dosage of the inhibitor.

The present invention further provides a method for selecting a therapy for a subject with a malignant cell comprising evaluating sensitivity said to a farnesyl protein transferase inhibitor; wherein said inhibitor is selected as the therapy if said cells are determined to be sensitive to the inhibitor. In an embodiment of the invention, after the inhibitor is selected as the therapy, the subject is administered a therapeutically effective dosage of the inhibitor optionally in association with a further therapeutic agent.

The scope of the present invention further includes a method of advertising a farnesyl protein transferase inhibitor therapy or a pharmaceutically acceptable composition thereof comprising promoting, to a target audience, the use of the inhibitor or pharmaceutical composition thereof for treating a patient or patient population whose tumors or cancerous conditions are mediated by malignant cells that exhibit increased expression of phosphorylated histone H2Ax following an initial treatment with said inhibitor.

The present invention also provides an article of manufacture comprising, packaged together, a pharmaceutical composition comprising a farnesyl protein transferase inhibitor and a pharmaceutically acceptable carrier; and a label stating or conveying (explicitly or otherwise) that the agent or pharmaceutical composition is indicated for treating patients having a tumor comprising malignant cells or a cancerous condition mediated by malignant cells that exhibit increased expression of phosphorylated histone H2Ax following an initial treatment with said inhibitor.

Also provided by the present invention is a method for manufacturing a farnesyl protein transferase inhibitor or a pharmaceutical composition thereof comprising combining, in a package, the inhibitor or pharmaceutical composition; and a label stating that the agent or pharmaceutical composition is indicated for treating patients having a tumor comprising malignant cells or a cancerous condition mediated by malignant cells that express increased levels of phosphorylated histone H2Ax following an initial treatment with said inhibitor.

### BRIEF DESCRIPTION OF THE FIGURES

FIG. 1. Western blot analysis of total histone H2Ax and phospho-histone H2Ax expression in breast and ovarian tumor cells following treatment with lonafarnib (336), DMSO or an inactive enantiomer of lonafarnib (337).

FIG. 2. Western blot analysis of total histone H2Ax and phospho-histone H2Ax expression in pancreatic and prostate tumor cells following treatment with lonafarnib (336), DMSO or an inactive enantiomer of lonafarnib (337).

FIG. 3. Western blot analysis of total histone H2Ax and phospho-histone H2Ax expression in colon and brain tumor cells following treatment with lonafarnib (336), DMSO or an inactive enantiomer of lonafarnib (337).

FIG. 4. Western blot analysis of phospho-histone H2Ax expression in a lonafarnib-sensitive human breast adenocarcinoma cell line, MCF-7, following treatment with DMSO, lonafarnib (336), inactive enantiomer of lonafarnib (337) or a structurally-unrelated FTI (SU-FTI).

### DETAILED DESCRIPTION OF THE INVENTION

The present invention provides a method for determining if a tumor is sensitive to an FTI. It has been discovered that malignant cells exhibiting sensitivity to an FTI will exhibit an increased level of phosphorylated histone H2Ax expression following contact with the FTI; whereas FTI resistant cells tend not to have increased levels of phosphorylated histone H2Ax. This characteristic of FTI sensitive and resistant malignant cells provides a rapid and convenient marker for FTI sensitivity. Using this marker, a doctor treating a subject with a tumor may make a rapid, convenient, individual prediction of whether the subject's tumor is FTI sensitive and, thus, whether an FTI treatment regimen is appropriate for that particular subject. This method is considerably more convenient than the alternative of monitoring tumor size and metastasis in the subject receiving FTI treatment over the course of weeks or months. Generally, the method includes making an initial determination of the level of phosphorylated histone H2Ax exhibited by cells of the subject's tumor or cancerous condition, then providing the patient with an initial course of treatment with the FTI, and then, again, determining the level of phosphorylated histone H2Ax exhibited by the cells. If the level of phosphorylated histone H2Ax increases following the initial FTI treatment, then the doctor may make the decision to continue the FTI treatment regimen.

The term “subject” or “patient” or the like includes animals such as mammals, including humans, canines, cats, cows, horses, rats, mice, rabbits, monkeys and apes.

The term “FPT” is farnesyl protein transferase. The term “FTI” is farnesyl protein transferase inhibitor.

In an embodiment of the invention, a cell is sensitive or responsive to a farnesyl protein transferase inhibitor (FTI) if its growth or survival or ability to metastasize is reduced to any detectable degree. In an embodiment of the invention, a cell is FTI sensitive if the IC50 for the FTI is less than about 1000 nM (e.g., 750 nM, 500 nM, 100 nM, 50 nM, 25 nM, 1 nM, 2 nM, or 3 nM or less) and the cell is FTI resistant if the IC50 is about 1000 nM or more.

### Biomarkers and Uses Thereof

The present invention comprises use of phosphorylated histone H2Ax as a marker for FTI sensitivity. HH2Ax is a known gene. In an embodiment of the invention, histone H2Ax comprises the amino acid sequence:

(SEQ ID NO: 1; optionally lacking the N-terminal methionine)
MSGRGKTGGK ARAKAKSRSS RAGLQFPVGR VHRLLRKGHY AERVGAGAPV
YLAAVLEYLT AEILELAGNA ARDNKKTRII PRHLQLAIRN DEELNKLLGG
VTIAQGGVLP NIQAVLLPKK TSATVGPKAP SGGKKATQAS QEY

See e.g., UniProt database accession no. P16104.

In an embodiment of the invention, phospho-histone H2AX or phosphor-histone H2Ax or phosphorylated histone H2Ax, or the like, comprises one or more phosphorylated amino acids. Such phosphorylated amino acids may be at any available position, for example, at any serine (e.g., serine 140 if histone H2Ax comprises an N-terminal methionine; serine 139 if the methionine is missing), threonine or tyrosine.

Determination that a cell expresses increased levels of phospho-histone H2Ax, following exposure to an FTI, is done, in an embodiment of the invention, relative to a cell of that type (e.g., from the same tumor) prior to contact with the FTI. In an embodiment of the invention, phospho-histone H2Ax expression is considered induced, following FTI treatment, when the expression is observed to increase to any detectable degree whatsoever (e.g., an increase of 1%, 5%, 10%, 25%, 50%, 75%, 100%, 150%, 200%, 300%, 400%, 500%). In an embodiment of the invention, phospho-histone H2Ax is considered induced, following FTI treatment, when it is observed to be present in an assayed cell (or cells) in an amount which is at least about 2.2 to about 6.5 times (e.g., at least about 2.3 times, at least about 2.5 times, at least about 3 times, at least about 3.5 times, at least about 4 times, at least about 4.5 times, at least about 5 times, at least about 5.5 times, at least about 6 times) the amount present prior to FTI treatment. In an embodiment of the invention, the levels of phospho-histone H2Ax expressed are normalized against the amount of total histone H2Ax when an evaluation is done (before and/or after FTI treatment). In an embodiment of the invention, normalization is performed by dividing the amount of phospho-histone H2Ax by the amount of total histone H2Ax.

In specific embodiments of the invention, FTI sensitivity of malignant cells in a tumor can be evaluated or determined by an in vivo method comprising, e.g., the steps of (a) obtaining a malignant cell from a tumor or which mediates a cancerous condition in a subject not yet treated with the FTI; (b) evaluating expression of phosphorylated histone H2Ax in one or more of the malignant cells; (c) treating the subject with the FTI; (d) again obtaining one or more of the malignant cells from the subject after treatment with the FTI; and (e) again evaluating expression of said phosphorylated histone H2Ax in the cells. In this method, the malignant cells in the tumor or which mediate the cancerous condition are determined to be sensitive to the FTI if expression of phosphorylated histone H2Ax is observed to increase following treatment with the FTI.

In another specific embodiment of the invention, FTI sensitivity of malignant cells in a tumor can be evaluated or determined by an in vitro method comprising, e.g., the steps of (a) obtaining and growing/culturing malignant cells from a tumor obtained from a subject not yet treated with the FTI; (b) evaluating expression of phosphorylated histone H2Ax in the malignant cells; (c) contacting the same or different malignant cells (e.g., other malignant cells derived from the initially grown or cultured cells) with the FTI; (d) evaluating expression of phosphorylated histone H2Ax in the cells following contact with the FTI. In this method, the malignant cells in the tumor are determined to be sensitive to the FTI if expression of phosphorylated histone H2Ax is observed to increase following contact with the FTI.

The method for evaluating or determining FTI sensitivity can be applied to several different methods including, e.g., a method for selecting a subject with a tumor for treatment, of said tumor, with an FTI; a method for identifying a subject with an FTI sensitive tumor; a method for treating a tumor with an FTI; or a method for selecting a therapy or a treatment for a subject with a tumor.

A method of selecting a subject for FTI therapy includes, for example, evaluating sensitivity of malignant cells in the subject's tumor or which mediate the subject's cancerous condition to the FTI (e.g., as discussed herein); wherein the subject is selected if said cells are determined to be FTI sensitive.

The method for evaluating or determining FTI sensitivity can also be applied to a method for identifying a subject with a tumor comprising malignant cells or with a cancerous condition mediated by malignant cells likely to be sensitive to an FTI comprising evaluating sensitivity of the malignant cells to the FTI (e.g., as discussed herein); wherein the subject is identified if said cells are determined to be FTI sensitive.

Methods for treating tumors may also make use of the methods discussed herein whereby tumor cells are evaluated for FTI sensitivity. For example, the present invention comprises a method for treating a tumor with an FTI comprising evaluating sensitivity of malignant cells in the tumor to the FTI (e.g., by a method discussed herein) and continuing treatment with the FTI or a pharmaceutical composition thereof if said cells are determined to be FTI sensitive.

The present invention further comprises a method for selecting a therapy for a subject with a tumor comprising evaluating sensitivity of malignant cells in the tumor to a FTI (e.g., by a method discussed herein); wherein the FTI is selected as the therapy if the cells are determined to be FTI sensitive.

Furthermore, the present invention comprises a method of advertising an FTI therapy or a pharmaceutically acceptable composition thereof by promoting, to a target audience, the use of the FTI or pharmaceutical composition thereof for treating a patient or patient population whose tumors exhibit increased expression of phosphorylated histone H2Ax following an initial treatment with said inhibitor. Such methods include advertisement by any medium whatsoever, including, print media, electronic media (e.g., internet websites and email) and video media (e.g., television commercials). Doctors or other medical professionals, having viewed such an advertisement, may then undertake an evaluation of the malignant cells in the tumor of a subject under their care for FTI sensitivity, using a method discussed herein.

The present invention further provides articles of manufacture including, packaged together, an FTI (e.g., of a pharmaceutical composition thereof) including a label stating that the FTI or pharmaceutical composition thereof is indicated for treating patients having a tumor which exhibits increased expression of phosphorylated histone H2Ax following an initial treatment with said inhibitor.

Additionally, the present invention includes a method for manufacturing an FTI or a pharmaceutical composition thereof by combining, in a package, the FTI or pharmaceutical composition thereof and a label stating that the agent or pharmaceutical composition is indicated for treating patients having a tumor that expresses increased levels of phosphorylated histone H2Ax following an initial treatment with said inhibitor. The present invention includes the product of any such method.

The methods for patient selection and treatment discussed herein may be supplemented with conventional methods for evaluating the progress of an anti-cancer course of treatment. For example, in general, the size and progress of cancer can be determined, by complete blood tests and metabolic panels, computed tomography (CT scan or CAT scan) X-ray, magnetic resonance imaging (MRI), visually in a surgical procedure, by thymidine PET scan (see e.g., Wells et al., Clin. Oncol. 8: 7-14 (1996)), or by combination PET/CT (fusion PET/CT); or other methods known in the art.

### Farnesyl Protein Transferase Inhibitors (FTIs)

The present invention comprises methods wherein a farnesyl protein transferase inhibitor (FTI) is administered to a patient. The term farnesyl protein transferase inhibitor or FTI includes any substance which inhibits FTI activity (e.g., farnesylation of ras or any other substrate of FPT; see e.g., Hightower et al., Biochem. J. (2001) 360: 625-631) to any detectable degree.

In an embodiment of the invention, an FTI is lonafarnib

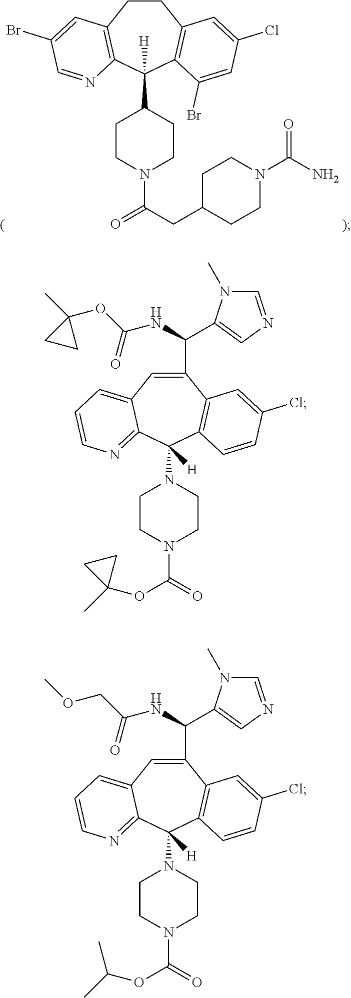

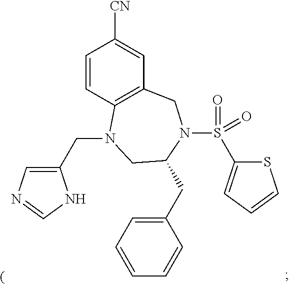

Hunt et al., J. Med. Chem. 43(20):3587-95 (2000); Dancey et al., Curr. Pharm. Des. 8:2259-2267 (2002); (R)-7-cyano-2,3,4,5-tetrahydro-1-(1H-imidazol-4-ylmethyl)-3-(phenylmethyl)-4-(2-thienylsulfonyl)-1H-1,4-benzodiazepine)); tipifarnib

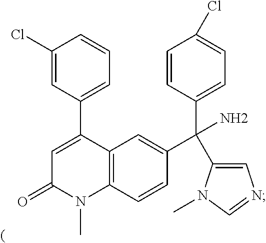

Garner et al., Drug Metab. Dispos. 30(7):823-30 (2002); Dancey et al., Curr. Pharm. Des. 8:2259-2267 (2002)), BMS-316810

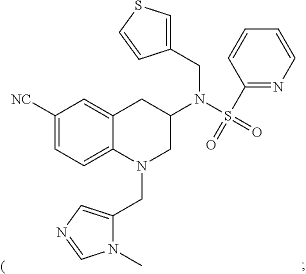

Lombardo et al., Bioorg. & Medi. Chem. Lett. (2005) 15(7):1895-1899),

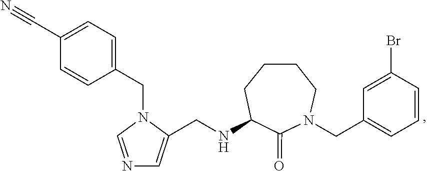

or manumycin A

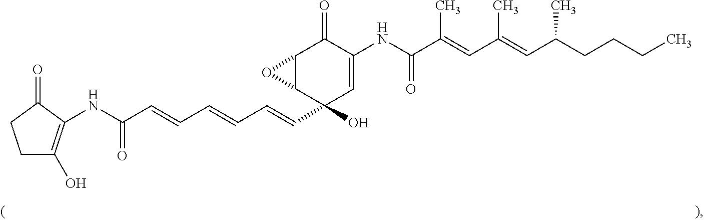

### FTI-276

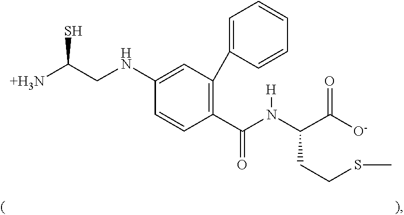

### or L-744832

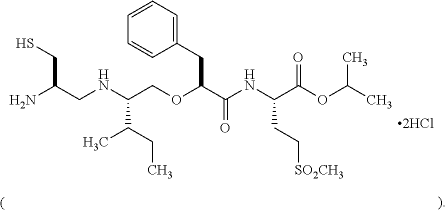

### Pharmaceutical Compositions, Dosage and Administration

The present invention comprises methods for treating or preventing any medical condition mediated by farnesylation with a farnesyl protein transferase (e.g., any hyperproliferative disease such as cancer) by administering an FTI or a pharmaceutical composition thereof comprising a pharmaceutically acceptable carrier.

In an embodiment of the invention, a medical condition treatable by a method of the present invention is breast tumor, an ovarian tumor, a pancreatic tumor, a prostate tumor, a colon tumor, a brain tumor, a urothelial tract tumor, a non-small cell lung tumor, chronic myelogenous leukemia (CML) or a taxane refractory/resistant non-small cell lung tumor. Medical conditions treatable include tumors and cancerous conditions. Cancerous conditions include conditions characterized by a malignancy, or the present of malignant cells in the body of a subject, but not necessarily by the presence of tumor. For example, a cancerous condition includes blood cancers such as leukemia (e.g., CML).

The present invention comprises evaluation of the sensitivity of malignant cells in a tumor in a patient, e.g., a breast or ovarian tumor or evaluation of a malignant cell which mediates a cancerous condition, e.g., a blood cell from a CML patient.

The terms: breast tumor, an ovarian tumor, a pancreatic tumor, a prostate tumor, a colon tumor, a brain tumor, a urothelial tract tumor, a non-small cell lung tumor, chronic myelogenous leukemia (CML) and a taxane refractory/resistant non-small cell lung tumor include any variety, subtype of stage of such a medical condition. For example, the term “breast tumor” includes ductal carcinomas, lobular carcinomas, Paget's disease of the nipple, BRCA1-mediated breast cancer and BRCA2-mediated breast cancer. The term “ovarian tumor” includes epithelial ovarian tumors, germ cell ovarian tumors and sex cord stromal ovarian tumors. The term “pancreatic tumor” includes exocrine tumors (e.g., ductal adenocarcinomas, cystic tumors, acinar cell tumors, and sarcomas), endocrine tumors (e.g., gastrinomas, insulinomas, somatostatinomas, VIPomas, and glucagonomas) and pancreatic lymphomas. For example, the term “prostate tumor” includes adenocarcinomas and prostatic intraepithelial neoplasia (PIN). The term “colon cancer” includes adenocarcinomas and leiomyosarcomas. The term “brain tumor” includes glioblastoma, glioma and astrocytoma. The term “urothelial tract tumor” includes bladder cancer, ureter cancer, renal pelvis cancer and renal calyces cancer. The term “non-small cell lung cancer” includes squamous cell carcinoma, adenocarcinoma and large-cell undifferentiated carcinoma. The term “chronic myelogenous leukemia” includes varieties of the disorder with and without the Philadelphia Chromosome (Ph¹) abnormality.

For general information concerning formulations, see, e.g., Gilman, et al., (eds.) (1990), The Pharmacological Bases of Therapeutics, 8th Ed., Pergamon Press; A. Gennaro (ed.), Remington's Pharmaceutical Sciences, 18th Edition, (1990), Mack Publishing Co., Easton, Pa.; Avis, et al., (eds.) (1993) Pharmaceutical Dosage Forms: Parenteral Medications Dekker, New York; Lieberman, et al., (eds.) (1990) Pharmaceutical Dosage Forms: Tablets Dekker, New York; and Lieberman, et al., (eds.) (1990), Pharmaceutical Dosage Forms: Disperse Systems Dekker, New York, Kenneth A. Walters (ed.) (2002) Dermatological and Transdermal Formulations (Drugs and the Pharmaceutical Sciences), Vol 119, Marcel Dekker. See also U.S. Pat. No. 6,632,455; and European patent no. 1039908.

Inert, pharmaceutically acceptable carriers used for preparing pharmaceutical compositions of FPT inhibitors described herein can be solid or liquid. Solid preparations include powders, tablets, dispersible granules, capsules, cachets and suppositories. The powders and tablets may, in an embodiment of the invention, comprise from about 5 to about 70% FTI. Solid carriers are known in the art, e.g., magnesium carbonate, magnesium stearate, talc, sugar, and/or lactose. Tablets, powders, cachets and capsules can, in an embodiment of the invention, be used as solid dosage forms suitable for oral administration.

In an embodiment of the invention, for preparing suppositories, a low melting wax such as a mixture of fatty acid glycerides or cocoa butter is first melted, and the FTI is dispersed homogeneously therein as by stirring. The molten homogeneous mixture is then poured into conveniently sized molds, allowed to cool and thereby solidify.

Liquid preparations include, in an embodiment of the invention, solutions, suspensions and emulsions. As an example may be mentioned water or water-propylene glycol solutions for parenteral injection. Liquid preparations may also include, in an embodiment of the invention, solutions for intranasal administration.

Aerosol preparations suitable for inhalation may include, in an embodiment of the invention, solutions and solids in powder form, which may be in combination with a pharmaceutically acceptable carrier, such as an inert compressed gas.

Also included in an embodiment of the invention are solid preparations which are intended for conversion, shortly before use, to liquid preparations for either oral or parenteral administration. Such liquid forms include, in an embodiment of the invention, solutions, suspensions and emulsions.

The FPT inhibitors described herein may also be deliverable, in an embodiment of the invention, transdermally. The transdermal compositions can take the form of creams, lotions, aerosols and/or emulsions and can be included in a transdermal patch of the matrix or reservoir type as are conventional in the art for this purpose.

In an embodiment of the invention, the FPT inhibitors are administered orally. In an embodiment of the invention, the pharmaceutical preparation is in unit dosage form. In such a form, the preparation is subdivided into unit doses containing appropriate quantities of FTI, e.g., an effective amount to achieve the desired purpose.

In an embodiment of the invention, the quantity of FTI in a unit dose of preparation is varied or adjusted from about 0.5 mg to 1000 mg, preferably from about 1 mg to 300 mg, more preferably 5 mg to 200 mg, according to the particular application.

In an embodiment of the invention, a therapeutically effective dosage or amount of any chemotherapeutic agent (e.g., as set forth herein) is, whenever possible, as set forth in the Physicians' Desk Reference 2003 (Thomson Healthcare; 57th edition (Nov. 1, 2002)) which is herein incorporated by reference or in the scientific literature.

The actual dosage employed may be varied depending upon the requirements of the patient and the severity of the condition being treated. Determination of the proper dosage for a particular situation is within the skill of the art. In an embodiment of the invention, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired.

In an embodiment of the invention, a therapeutically effective amount of an FPT inhibitor (e.g., lonafarnib) is about 200 mg BID (twice daily).

In a combination therapy embodiment of the present invention, a low dosage regimen of the FPT inhibitors is, e.g., oral administration of an amount in the range of from 1.4 to 400 mg/day, e.g., 1.4 to 350 mg/day, or 3.5 to 70 mg/day, e.g., with a BID dosing schedule. A particularly low dosage range can, in an embodiment of the invention, be 1.4 to 70 mg/day.

In an embodiment of the invention, a therapeutically effective dosage of lonafarnib and a taxane, such as paclitaxel, when co-administered, is as follows: lonafarnib (e.g., capsules taken orally) twice daily with food at 50 mg, 75 mg, 100 mg or 200 mg with the paclitaxel (e.g., administered intravenously) every 3 weeks at 135 mg/m² or 175 mg/m² over 3 h (see e.g., Khuri et al., Clinical Cancer Research 10: 2968-2976 (2004)).

In an embodiment of the invention, a therapeutically effective dosage of lonafarnib and docetaxel, temozolomide or anastrazole is about 200 mg BID lonafarnib and the approved dosage of docetaxel, temozolomide or anastrazole. In an embodiment of the invention, the docetaxel regimen is for treatment of prostate cancer.

In an embodiment, a therapeutically effective dosage of any anti-IGF1R antibody (e.g., 19D12/15H12 LCF/HCA), which may be administered in association with an FPT inhibitor is in the range of about 0.3 mg/kg (body weight) to about 20 mg/kg (e.g., 0.3 mg/kg, 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg or 20 mg/kg) per day (e.g., 1 time, 2 times or 3 times per week).

In an embodiment of the invention, any antineoplastic agent used with an FPT inhibitor is administered in its normally prescribed dosages during the treatment cycle (i.e., the antineoplastic agents are administered according to the standard of practice for the administration of these drugs).

In an embodiment of the invention, lonafarnib is administered to treat advanced urothelial tract cancer at 150 mg in the morning and 100 mg in the evening along with gemcitabine at 1000 mg/m² on day 1, 8 and 15 per 28-day cycle (Theodore et al. Eur. J. Cancer (2005) 41(8):1150-7).

In an embodiment of the invention, lonafarnib is administered to treat solid cancers (e.g., non-small cell lung cancer) p.o., twice daily (BID) on continuously scheduled doses of 100 mg or 125 mg or 150 mg in combination with intravenous paclitaxel at doses of 135 mg/m² or 175 mg/m² administered over 3 hours on day 8 of every 21 day cycle (Khuri et al., Clin. Cancer Res. (2004) 10(9):2968-76).

In an embodiment of the invention, lonafarnib is administered to treat chronic myelogenous leukemia (CML) at 200 mg orally twice daily (Borthakur et al., Cancer (2006)106(2):346-52).

In an embodiment of the invention, lonafarnib is administered to treat taxane-refractory/resistant non-small cell lung carcinoma at 100 mg orally twice per day beginning on Day 1 and paclitaxel 175 mg/m² intravenously over 3 hours on Day 8 of each 21-day cycle (Kim et al., Cancer (2005) 104(3):561-9).

### Further Chemotherapeutics

The scope of the present invention comprises methods for treating patients or subjects comprising a disease mediated by farnesyl protein transferase comprising administering an FTI or a pharmaceutical composition thereof to said patient optionally in association with a further chemotherapeutic agent. A further chemotherapeutic agent comprises any agent that elicits a beneficial physiological response in an individual to which it is administered; for example, wherein the agent alleviates or eliminates disease symptoms or causes within the subject to which it is administered. A further chemotherapeutic agent includes any anti-cancer chemotherapeutic agent. An anti-cancer therapeutic agent is any agent that, for example, alleviates or eliminates symptoms or causes of cancer in the subject to which it is administered.

The term “in association with” indicates that an FTI and another therapeutic agent (e.g., paclitaxel) can be formulated into a single composition for simultaneous delivery or formulated separately into two or more compositions (e.g., a kit). Furthermore, each component can be administered to a subject at a different time than when the other component is administered; for example, each administration may be given non-simultaneously (e.g., separately or sequentially) at several intervals over a given period of time. Moreover, the separate components may be administered to a subject by the same or by a different route.

The present invention includes embodiments comprising administering, to a patient, an FTI in association with an anti-IGF1R antibody or antigen-binding fragment including, e.g., 15H12/19D12 LCA, 15H12/19D12 LCB, 15H12/19D12 LCC, 15H12/19D12 LCD, 15H12/19D12 LCE, 15H12/19D12 LCF, 15H12/19D12 HCA or 15H12/19D12 HCB or any combination thereof (e.g., a full antibody comprising LCC and HCA or LCF and HCA).

Dotted, underscored type encodes the signal peptide. Solid underscored type encodes the CDRs. Plain type encodes the framework regions. Most preferably, the antibody chains are mature fragments which lack the signal peptide.

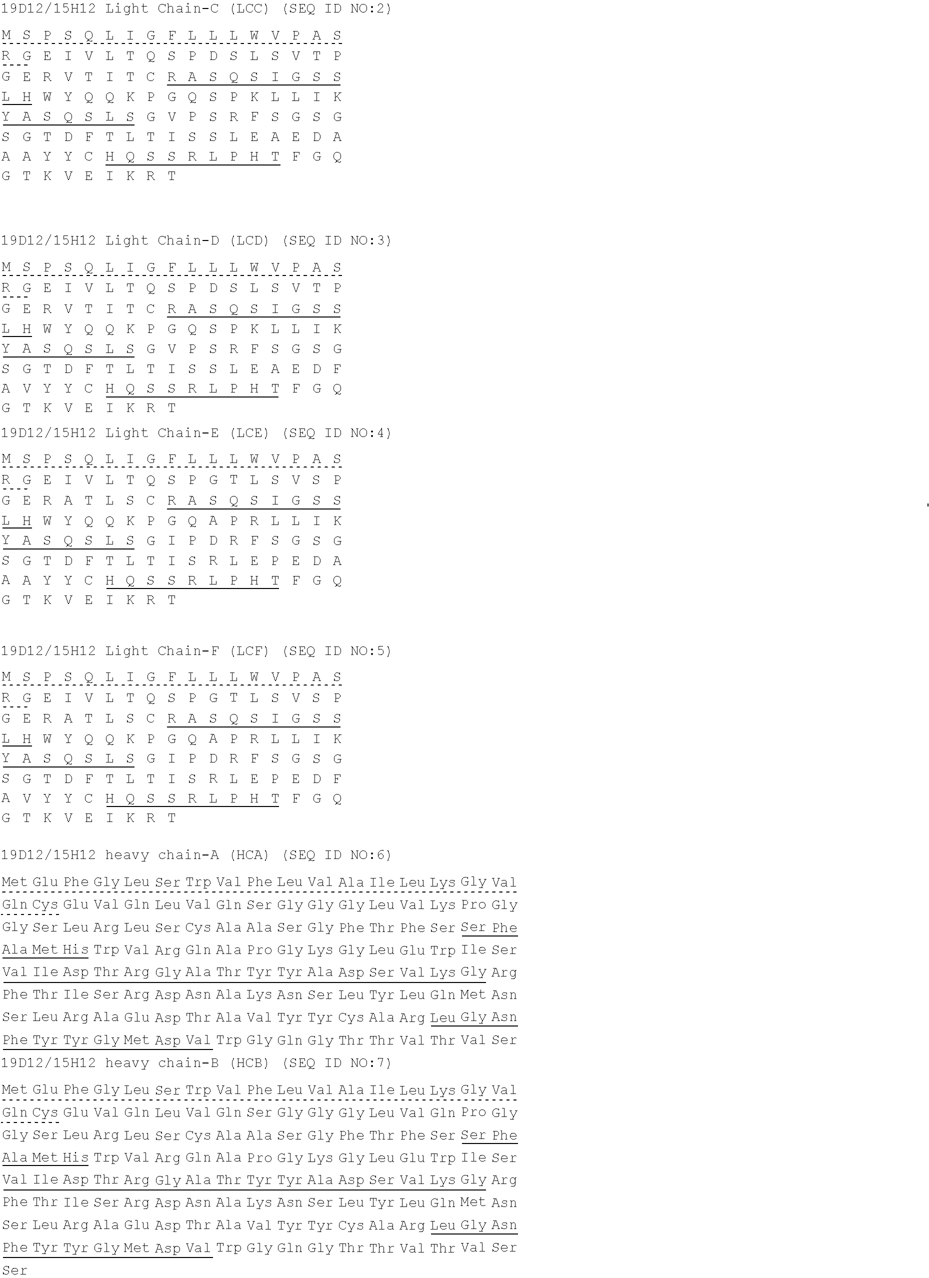

See international application publication no. WO03/100008.

In an embodiment of the invention, an FTI is administered in association with any aurora kinase inhibitor, KSP inhibitor, HSP90 inhibitor, JAK inhibitor, any STAT inhibitiors, any histone deacetylase inhibitor, any MEKK inhibitor, any MEK inhibitor, any MAPK inhibitor, rAd-p53 or any other p53 gene therapy agent, any rheb inhibitor, any TSC-1 agonist and/or any TSC-2 agonists, any AKT inhibitor, any mTOR inhibitor, any S6 kinase inhibitor, any MDM2 inhibitor, any MDM2 inhibitor in association with any p53 enhancing therapies including p53 gene therapy, rAd-p53.

In an embodiment of the invention, an FTI is administered in association with VX680, AZD1152, 17-AAG, 17-DMAG, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-1-TM-601, ALT-110, BIO 140, CC 8490, cilengitide, gimatecan, IL13-PE38QQR, INO 1001, IPdR, KRX-0402, lucanthone, LY 317615, neuradiab, vitespan, Rta 744, Sdx 102, talampanel, atrasentan or Xr 311.

In an embodiment of the invention, an FTI is administered in association with romidepsin (FK-228;

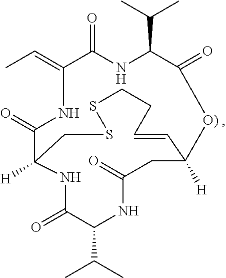

### ADS-100380,

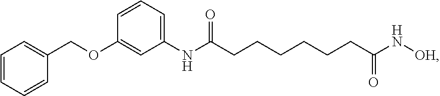

### CG-781

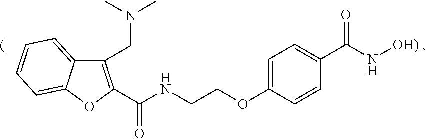

### CG-1521

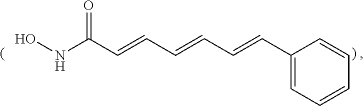

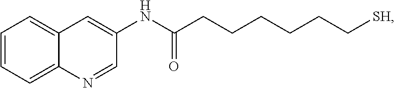

### SB-556629

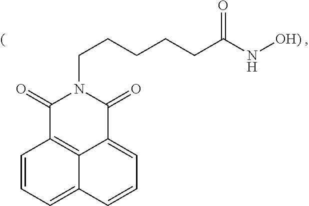

chlamydocin

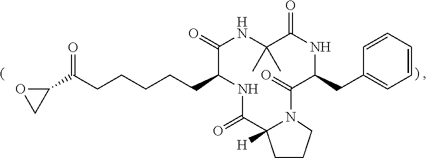

### JNJ-16241199

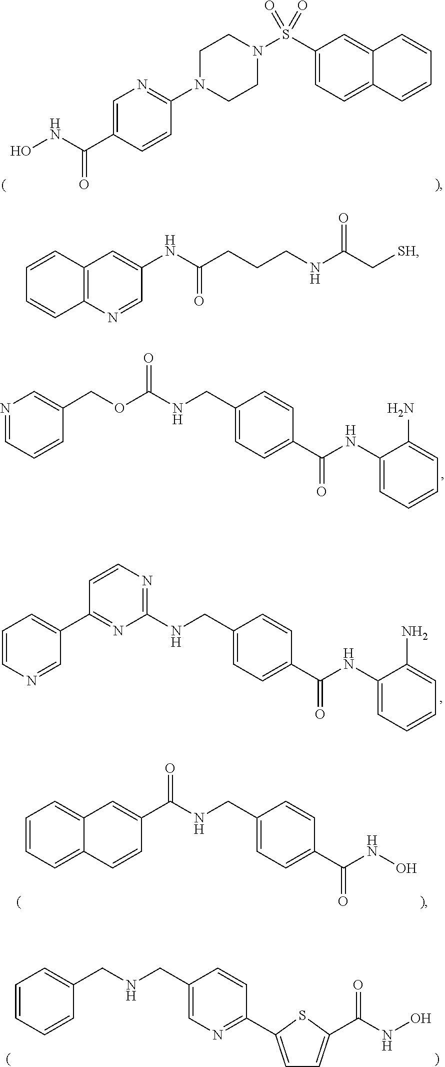

or vorinostat (SAHA;

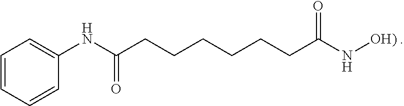

In an embodiment of the invention, an FTI is administered in association with etoposide (VP-16;

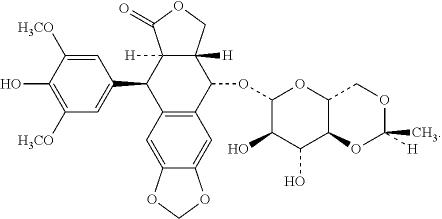

In an embodiment of the invention, an FTI is administered in association with gemcitabine

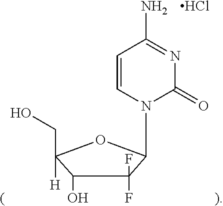

In an embodiment of the invention, an FTI is administered in association with any compound disclosed in published U.S. patent application no. U.S. 2004/0209878A1 (e.g., comprising a core structure represented by

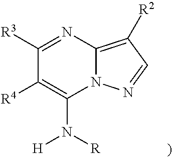

or doxorubicin

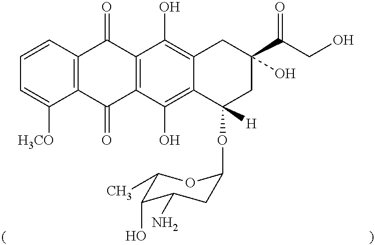

including Caelyx or Doxil® (doxorubicin HCl liposome injection; Ortho Biotech Products L.P; Raritan, N.J.). Doxil® comprises doxorubicin in STEALTH® liposome carriers which are composed of N-(carbonyl-methoxypolyethylene glycol 2000)-1,2-distearoyl-sn-glycero-3-phosphoethanolamine sodium salt (MPEG-DSPE); fully hydrogenated soy phosphatidylcholine (HSPC), and cholesterol.

In an embodiment of the invention, an FTI is administered in association with 5′-deoxy-5-fluorouridine

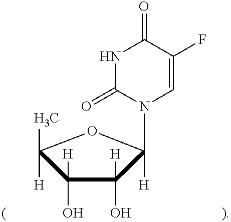

In an embodiment of the invention, an FTI is administered in association with vincristine

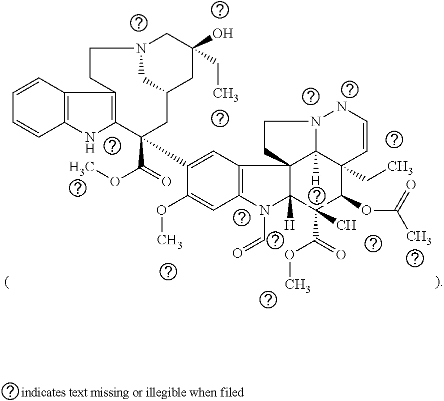

In an embodiment of the invention, an FTI is administered in association with temozolomide

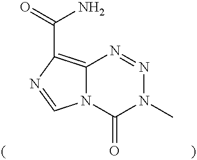

any CDK inhibitor such as ZK-304709, Seliciclib (R-roscovitine)

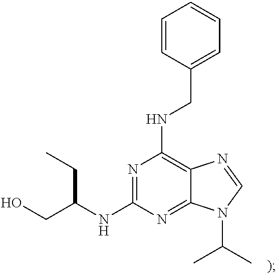

any MEK inhibitor such as PD0325901

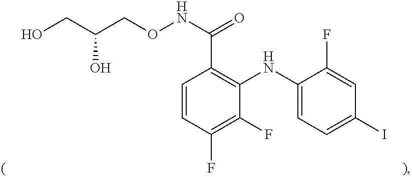

AZD-6244; capecitabine (5′-deoxy-5-fluoro-N-[(pentyloxy) carbonyl]-cytidine); or L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate

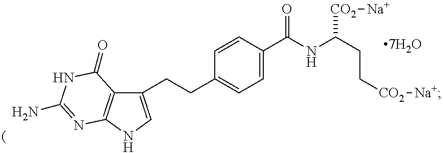

Pemetrexed disodium heptahydrate).

In an embodiment of the invention, an FTI is administered in association with camptothecin

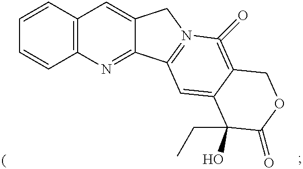

Stork et al., J. Am. Chem. Soc. 93(16): 4074-4075 (1971); Beisler et al., J. Med. Chem. 14(11): 1116-1117 (1962)), irinotecan (

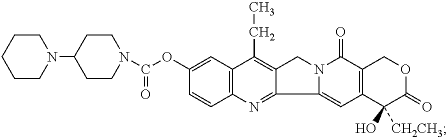

sold as Camptosar®; Pharmacia & Upjohn Co.; Kalamazoo, Mich.) or PEG-labeled irinotecan.

In an embodiment of the invention, an FTI is administered in association with the FOLFOX regiment (oxaliplatin

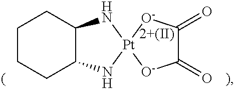

together with infusional fluorouracil

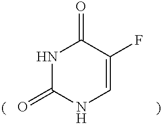

and folinic acid

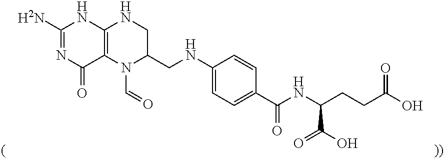

(Chaouche et al., Am. J. Clin. Oncol. 23(3):288-289 (2000): de Gramont et al., J. Clin. Oncol. 18(16):2938-2947 (2000)).

In an embodiment of the invention, an FTI is administered in association with an antiestrogen such as

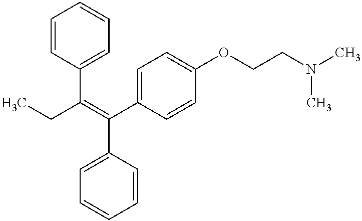

(tamoxifen; sold as Nolvadex® by AstraZeneca Pharmaceuticals LP; Wilmington, Del.) or

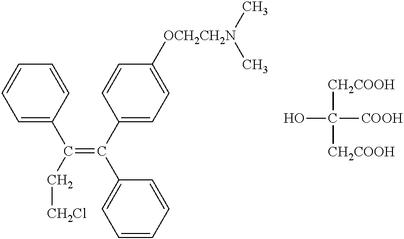

(toremifene citrate; sold as Fareston® by Shire US, Inc.; Florence, Ky.).

In an embodiment of the invention, an FTI is administered in association with an aromatase inhibitor such as

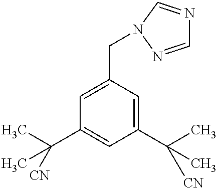

(anastrazole; sold as Arimidex® by AstraZeneca Pharmaceuticals LP; Wilmington, Del.),

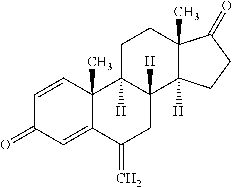

(exemestane; sold as Aromasin® by Pharmacia Corporation; Kalamazoo, Mich.) or

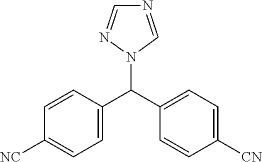

(letrozole; sold as Femara® by Novartis Pharmaceuticals Corporation; East Hanover, N.J.).

In an embodiment of the invention, an FTI is administered in association with an estrogen such as DES (diethylstilbestrol),

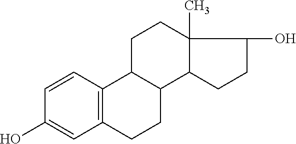

(estradiol; sold as Estrol® by Warner Chilcott, Inc.; Rockaway, N.J.) or conjugated estrogens (sold as Premarin® by Wyeth Pharmaceuticals Inc.; Philadelphia, Pa.).

In an embodiment of the invention, an FTI is administered in association with anti-angiogenesis agents including bevacizumab (Avastin™ Genentech; San Francisco, Calif.), the anti-VEGFR-2 antibody IMC-1C11, other VEGFR inhibitors such as: CHIR-258

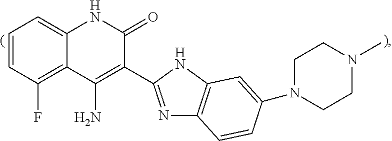

any of the inhibitors set forth in WO2004/13145 (e.g., comprising the core structural formula:

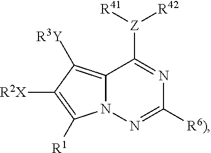

WO2004/09542 (e.g., comprising the core structural formula:

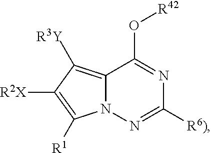

WO00/71129 (e.g., comprising the core structural formula:

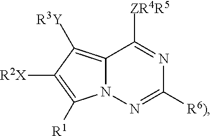

WO2004/09601 (e.g., comprising the core structural formula:

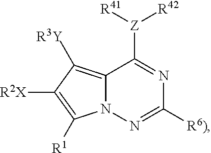

WO2004/01059 (e.g., comprising the core structural formula:

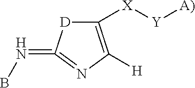

WO01/29025 (e.g., comprising the core structural formula:

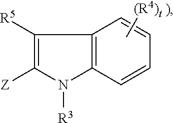

WO02/32861 (e.g., comprising the core structural formula:

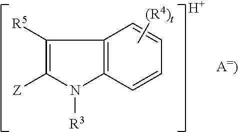

or set forth in WO03/88900 (e.g., comprising the core structural formula

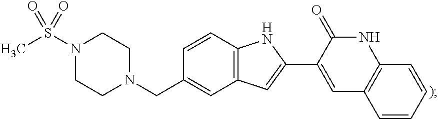

3-[5-(methylsulfonylpiperadinemethyl)-indolyl]-quinolone; Vatalanib

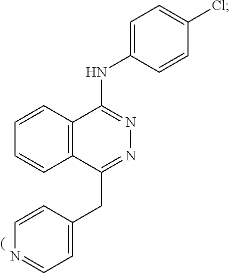

### PTK/ZK; CPG-79787; ZK-222584), AG-013736

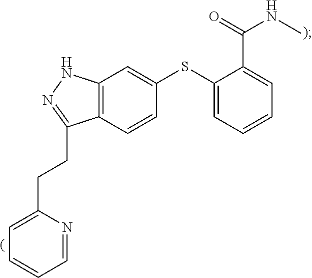

and the VEGF trap (AVE-0005), a soluble decoy receptor comprising portions of VEGF receptors 1 and 2.

In an embodiment of the invention, an FTI is administered in association with a LHRH (Lutenizing hormone-releasing hormone) agonist such as the acetate salt of [D-Ser(But)6,Azgly 10] (pyro-Glu-His-Trp-Ser-Tyr-D-Ser(But)-Leu-Arg-Pro-Azgly-NH₂ acetate [C₅₉H₈₄N₁₈O₁₄.(C₂H₄O₂)ₓ where x=1 to 2.4];

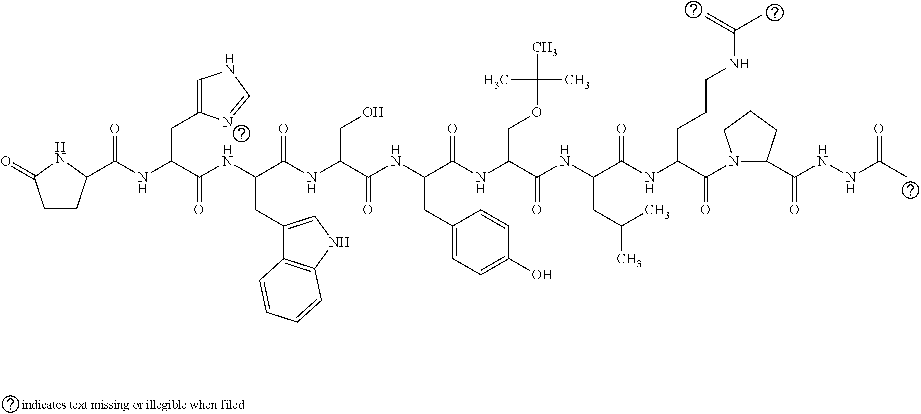

(goserelin acetate; sold as Zoladex® by AstraZeneca UK Limited; Macclesfield, England),

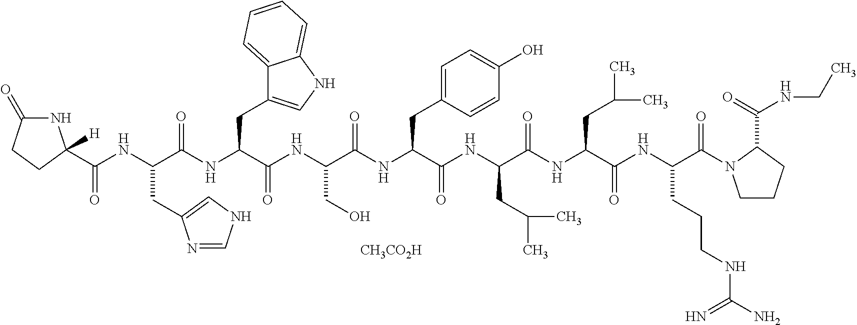

(leuprolide acetate; sold as Eligard® by Sanofi-Synthelabo Inc.; New York, N.Y.) or

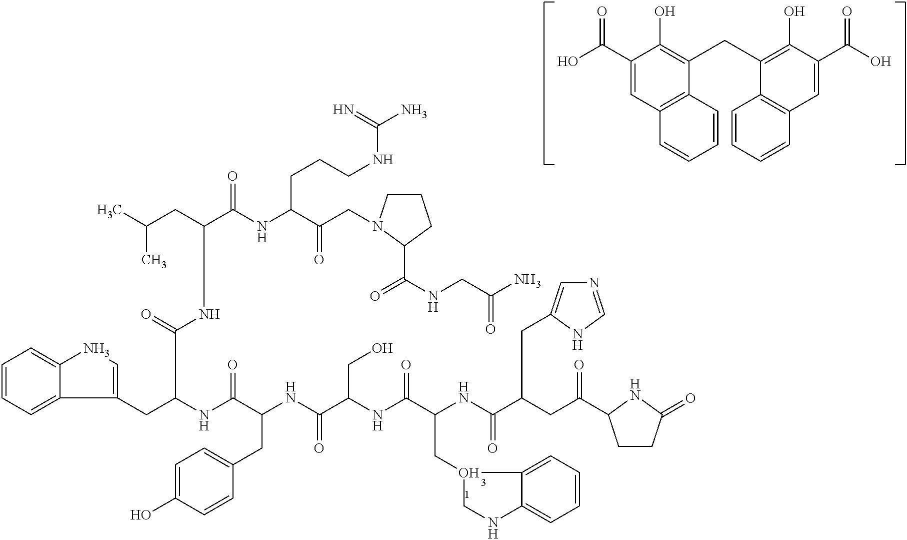

(triptorelin pamoate; sold as Trelstar® by Pharmacia Company, Kalamazoo, Mich.).

In an embodiment of the invention, an FTI is administered in association with sunitinib or sunitinib malate

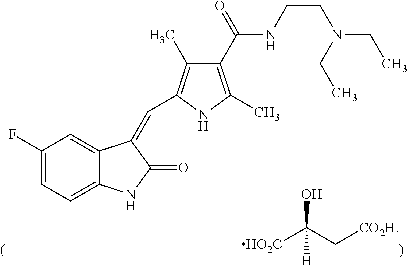

In an embodiment of the invention, an FTI is administered in association with a progestational agent such as

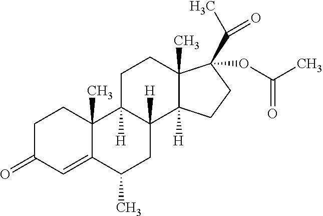

(medroxyprogesterone acetate; sold as Provera® by Pharmacia & Upjohn Co.; Kalamazoo, Mich.),

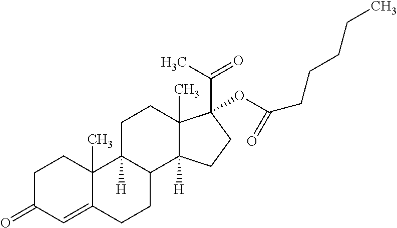

(hydroxyprogesterone caproate; 17-((1-Oxohexyl)oxy)pregn-4-ene-3,20-dione;), megestrol acetate or progestins.

In an embodiment of the invention, an FTI is administered in association with selective estrogen receptor modulator (SERM) such as

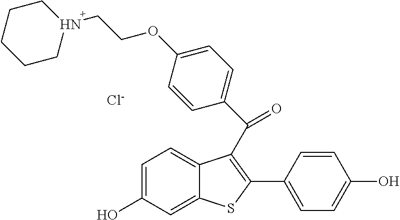

(raloxifene; sold as Evista® by Eli Lilly and Company; Indianapolis, Ind.).

In an embodiment of the invention, an FTI is administered in association with an anti-androgen including, but not limited to:

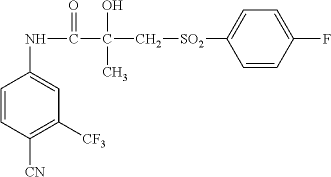

(bicalutamide; sold at CASODEX® by AstraZeneca Pharmaceuticals LP; Wilmington, Del.);

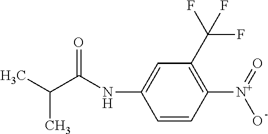

(flutamide; 2-methyl-N-[4-nitro-3 (trifluoromethyl)phenyl]propanamide; sold as Eulexin® by Schering Corporation; Kenilworth, N.J.);

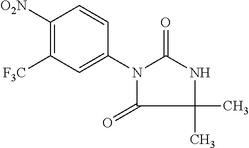

(nilutamide; sold as Nilandron® by Aventis Pharmaceuticals Inc.; Kansas City, Mo.) and

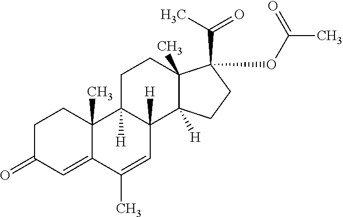

(Megestrol acetate; sold as Megace® by Bristol-Myers Squibb).

In an embodiment of the invention, an FTI is administered in association with one or more inhibitors which antagonize the action of the EGF Receptor or HER2, including, but not limited to, CP-724714

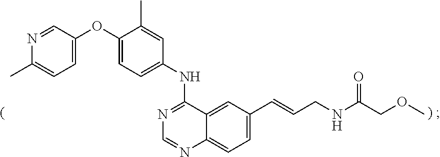

### TAK-165

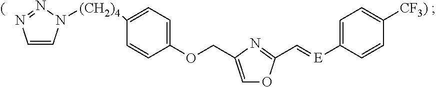

### HKI-272

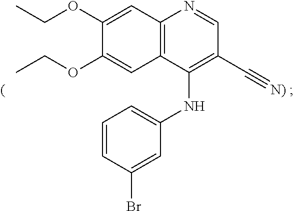

### OSI-774

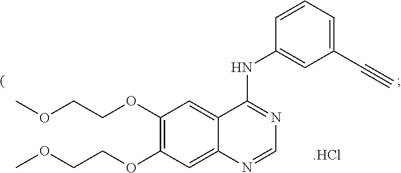

erlotinib, Hidalgo et al., J. Clin. Oncol. 19(13): 3267-3279 (2001)), Lapatanib

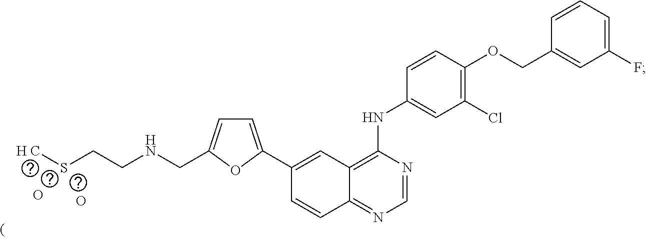

GW2016; Rusnak et al., Molecular Cancer Therapeutics 1:85-94 (2001); N-{3-Chloro-4-[(3-fluorobenzyl)oxy]phenyl}-6-[5-({[2-(methylsulfonyl)ethyl]amino}methyl)-2-furyl]-4-quinazolinamine; PCT Application No. WO99/35146), Canertinib (CI-1033;

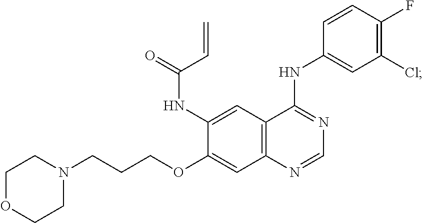

Erlichman et al., Cancer Res. 61(2):739-48 (2001); Smaill et al., J. Med. Chem. 43(7):1380-97 (2000)), ABX-EGF antibody (Abgenix, Inc.; Freemont, Calif.; Yang et al., Cancer Res. 59(6):1236-43 (1999); Yang et al., Crit Rev Oncol Hematol. 38(1):17-23 (2001)), erbitux (U.S. Pat. No. 6,217,866; IMC-C225, cetuximab; Imclone; New York, N.Y.), EKB-569

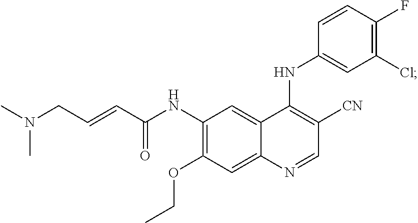

Wissner et al., J. Med. Chem. 46(1): 49-63 (2003)), PKI-166

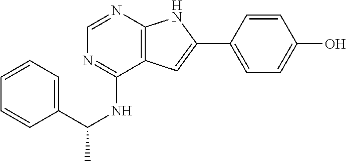

CGP-75166), GW-572016, any anti-EGFR antibody and any anti-HER2 antibody.

In an embodiment of the invention, an FTI is administered in association with

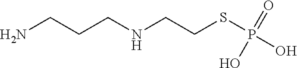

### (Amifostine);

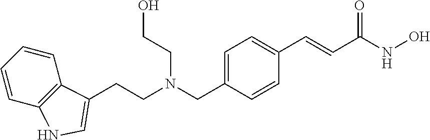

### (NVP-LAQ824; Atadja et al., Cancer Research 64: 689-695 (2004)),

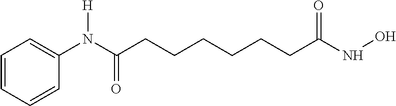

(suberoyl analide hydroxamic acid),

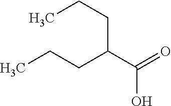

(Valproic acid; Michaelis et al., Mol. Pharmacol. 65:520-527 (2004)),

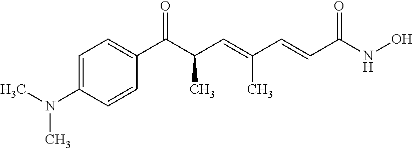

(trichostatin A),

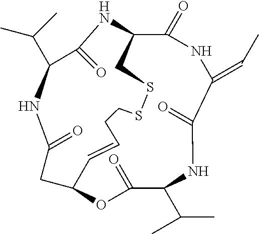

### (FK-228; Furumai et al., Cancer Research 62: 4916-4921 (2002)),

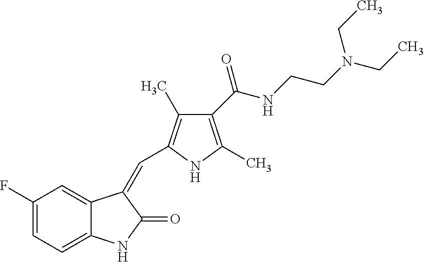

(SU11248; Mendel et al., Clin. Cancer Res. 9(1):327-37 (2003)),

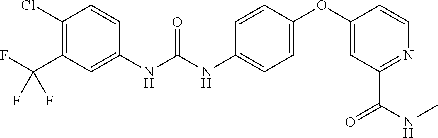

(BAY43-9006; sorafenib),

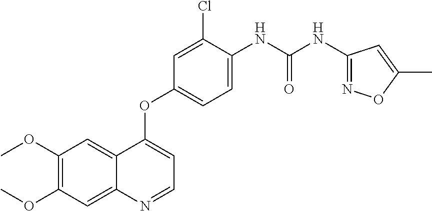

### (KRN951),

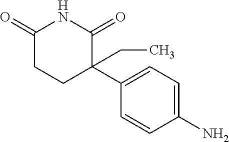

### (Aminoglutethimide);

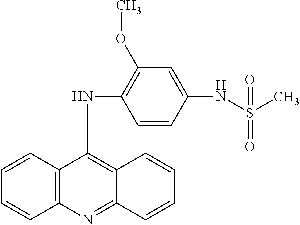

### (Amsacrine);

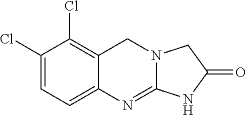

### (Anagrelide);

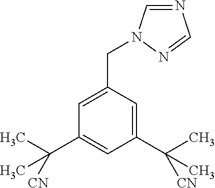

(Anastrozole; sold as Arimidex by AstraZeneca Pharmaceuticals LP; Wilmington, Del.); Asparaginase; Bacillus Calmette-Guerin (BCG) vaccine (Gamido et al., Cytobios. 90(360):47-65 (1997));

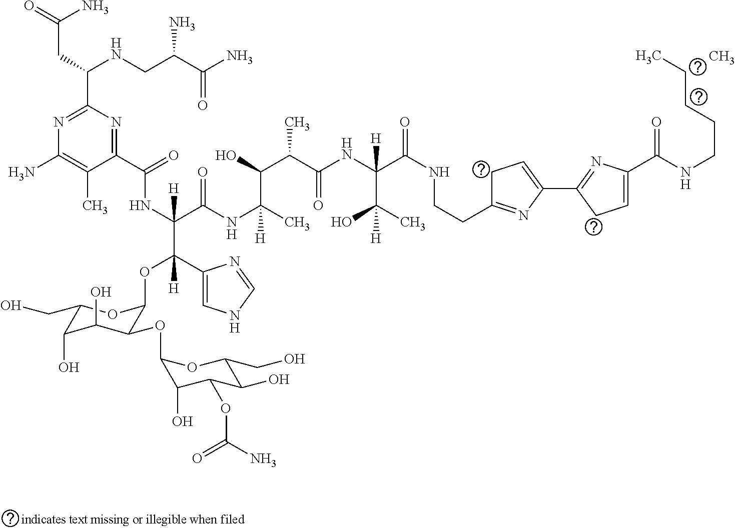

### (Bleomycin);

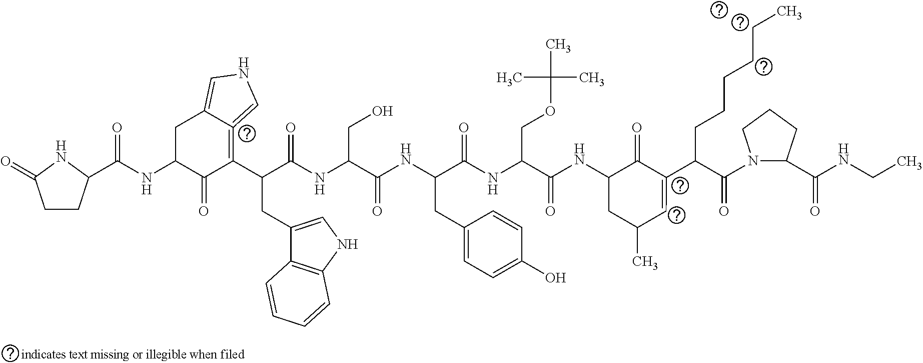

### (Buserelin);

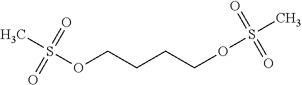

(Busulfan; 1,4-butanediol, dimethanesulfonate; sold as Busulfex® by ESP Pharma, Inc.; Edison, N.J.);

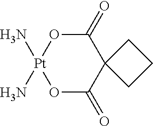

(Carboplatin; sold as Paraplatin® by Bristol-Myers Squibb; Princeton, N.J.)

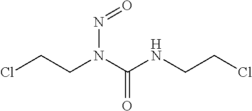

### (Carmustine);

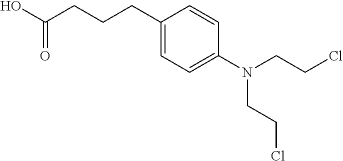

### (Chlorambucil);

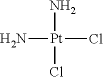

### (Cisplatin);

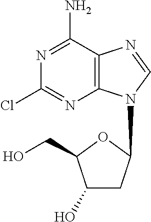

### (Cladribine);

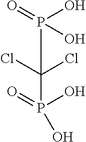

### (Clodronate);

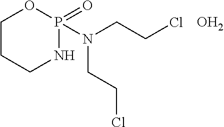

### (Cyclophosphamide);

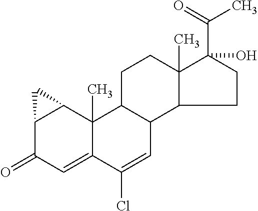

### (Cyproterone);

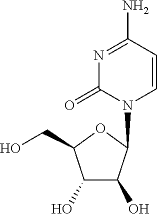

### (Cytarabine);

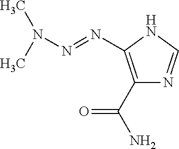

### (Dacarbazine);

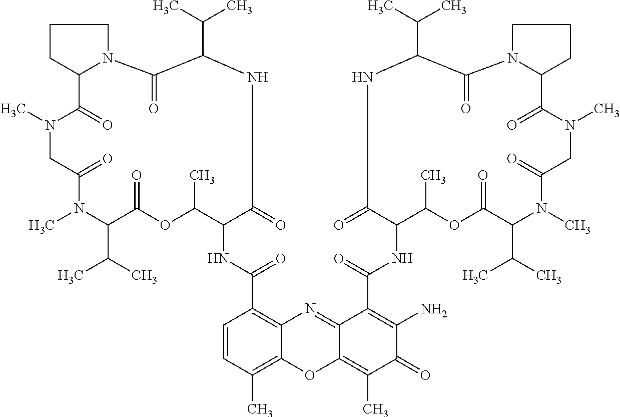

### (Dactinomycin);

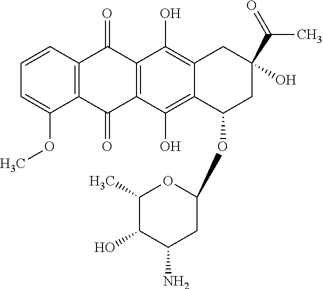

### (Daunorubicin);

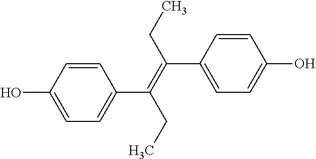

### (Diethylstilbestrol);

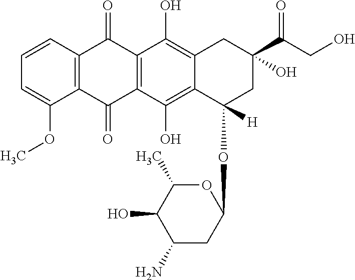

### (Epirubicin);

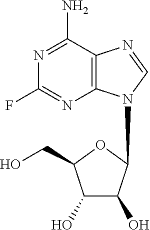

### (Fludarabine);

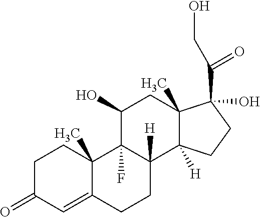

### (Fludrocortisone);

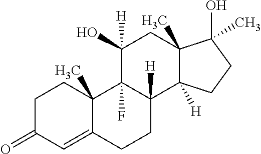

### (Fluoxymesterone);

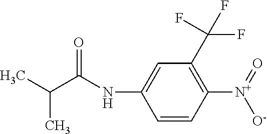

### (Flutamide);

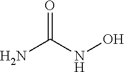

### (Hydroxyurea);

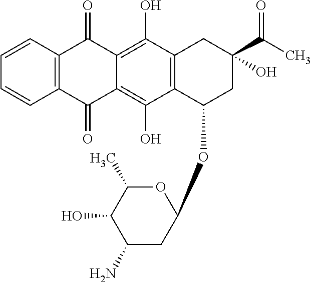

### (Idarubicin);

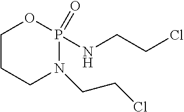

### (Ifosfamide);

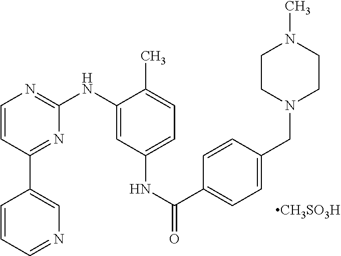

(Imatinib; sold as Gleevec® by Novartis Pharmaceuticals Corporation; East Hanover, N.J.);

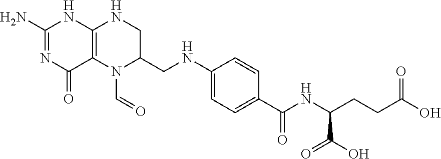

### (Leucovorin);

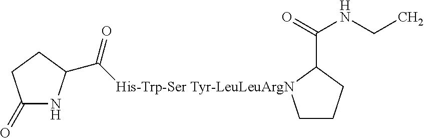

### (Leuprolide);

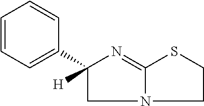

### (Levamisole);

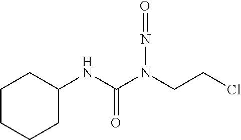

### (Lomustine);

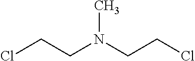

### (Mechlorethamine);

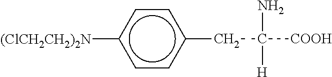

(Melphalan; sold as Alkeran® by Celgene Corporation; Warren, N.J.);

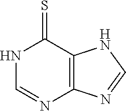

### (Mercaptopurine);

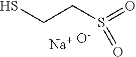

### (Mesna);

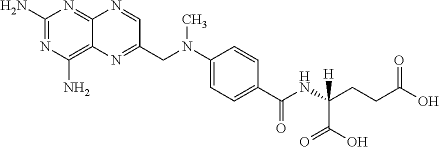

### (Methotrexate);

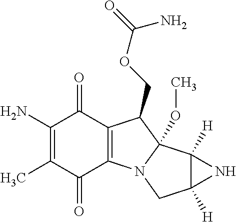

### (Mitomycin);

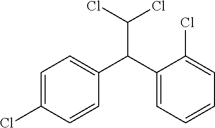

### (Mitotane);

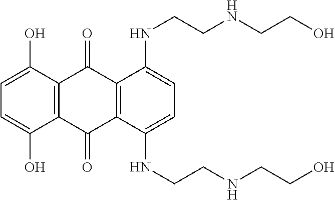

### (Mitoxantrone);

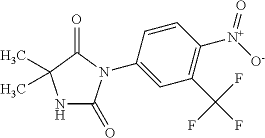

(Nilutamide); octreotide

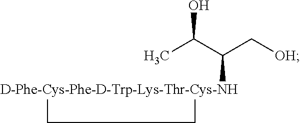

Katz et al., Clin Pharm. 8(4):255-73 (1989); sold as Sandostatin LAR® Depot; Novartis Pharm. Corp; E. Hanover, N.J.); edotreotide (yttrium-90 labeled or unlabeled); oxaliplatin

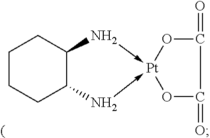

sold as Eloxatin™ by Sanofi-Synthelabo Inc.; New York, N.Y.);

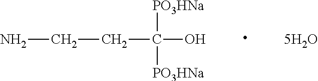

(Pamidronate; sold as Aredia® by Novartis Pharmaceuticals Corporation; East Hanover, N.J.);

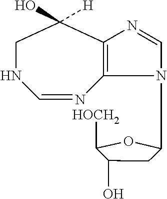

(Pentostatin; sold as Nipent® by Supergen; Dublin, Calif.);

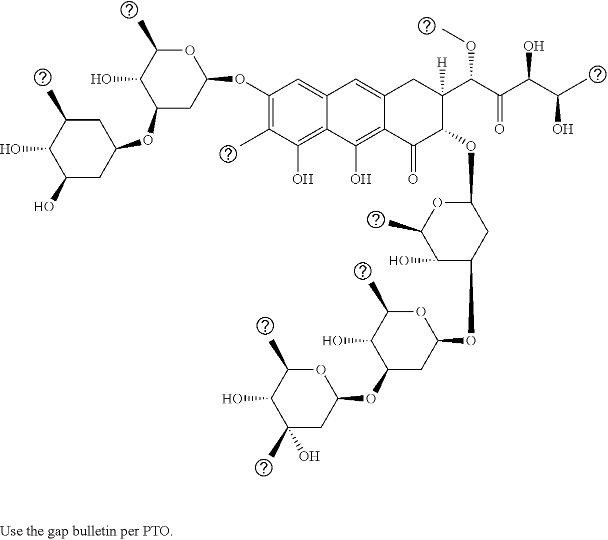

### (Plicamycin);

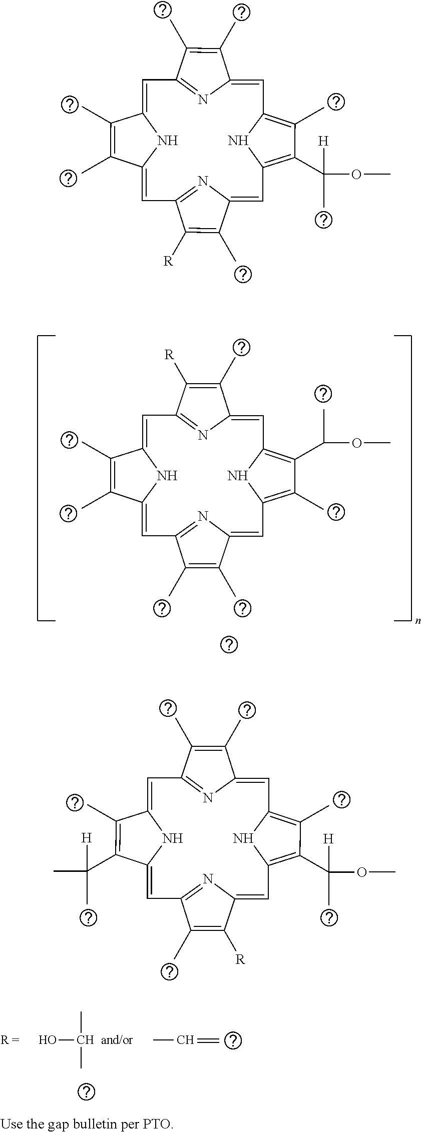

(Porfimer; sold as Photofrin® by Axcan Scandipharm Inc.; Birmingham, Ala.);

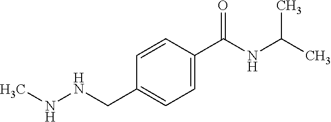

### (Procarbazine);

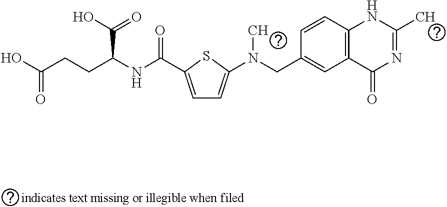

(Raltitrexed); Rituximab (sold as Rituxan® by Genentech, Inc.; South San Francisco, Calif.);

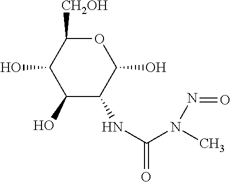

### (Streptozocin);

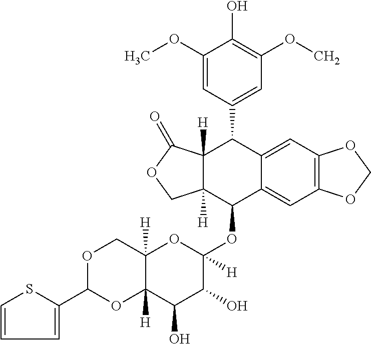

### (Teniposide);

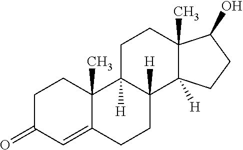

### (Testosterone);

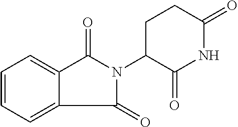

### (Thalidomide);

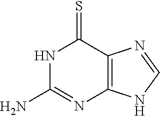

### (Thioguanine);

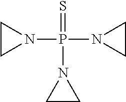

### (Thiotepa);

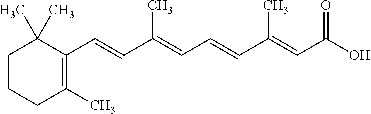

### (Tretinoin);

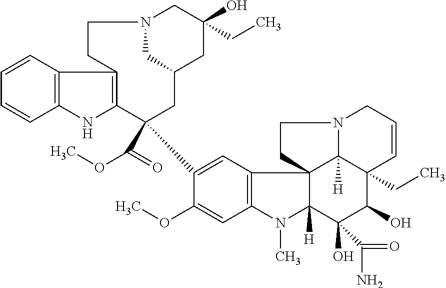

(Vindesine) or 13-cis-retinoic acid

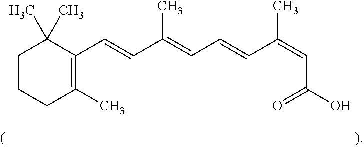

In an embodiment of the invention, an FTI is administered in association with one or more of any of: phenylalanine mustard, uracil mustard, estramustine, altretamine, floxuridine, 5-deooxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin, diftitox, gefitinib, bortezimib, paclitaxel, docetaxel, epithilone B, BMS-247550 (see e.g., Lee et al., Clin. Cancer Res. 7:1429-1437 (2001)), BMS-310705, droloxifene (3-hydroxytamoxifen), 4-hydroxytamoxifen, pipendoxifene, ERA-923, arzoxifene, fulvestrant, acolbifene, lasofoxifene (CP-336156), idoxifene, TSE-424, HMR-3339, ZK186619, topotecan, PTK787/ZK 222584 (Thomas et al., Semin Oncol. 30(3 Suppl 6):32-8 (2003)), the humanized anti-VEGF antibody Bevacizumab, VX-745 (Haddad, Curr Opin. Investig. Drugs 2(8):1070-6 (2001)), PD 184352 (Sebolt-Leopold, et al. Nature Med. 5: 810-816 (1999)), any mTOR inhibitor, rapamycin

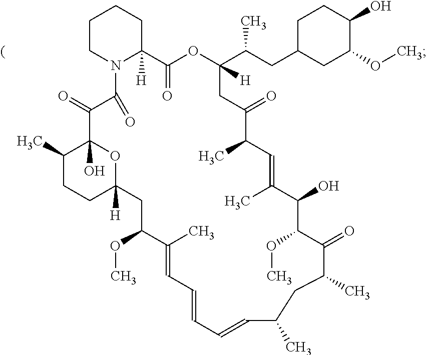

sirolimus), 40-O-(2-hydroxyethyl)-rapamycin, CCI-779

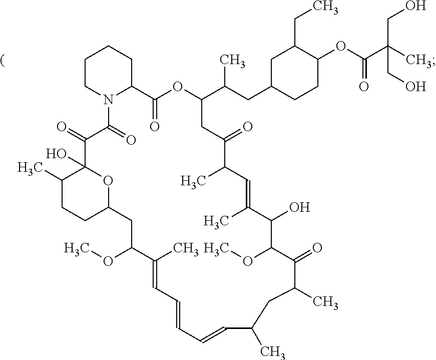

temsirolimus; Sehgal et al., Med. Res. Rev., 14:1-22 (1994); Elit, Curr. Opin. Investig. Drugs 3(8):1249-53 (2002)), AP-23573

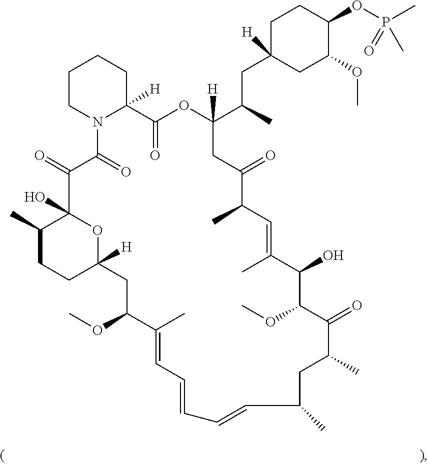

### RAD001

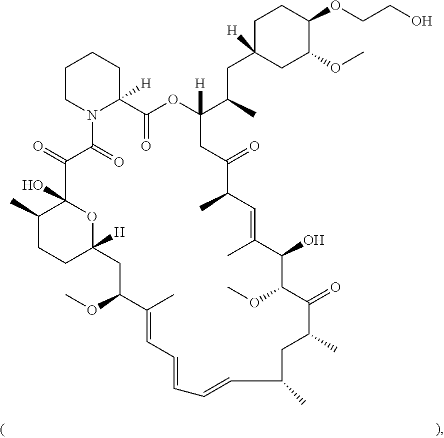

### ABT-578

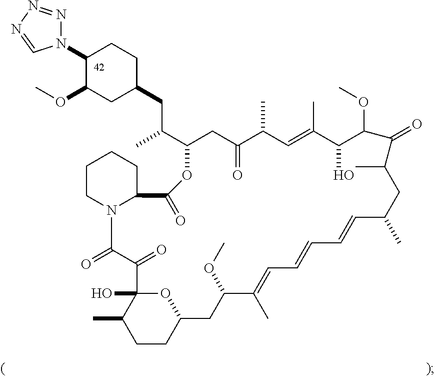

### BC-210

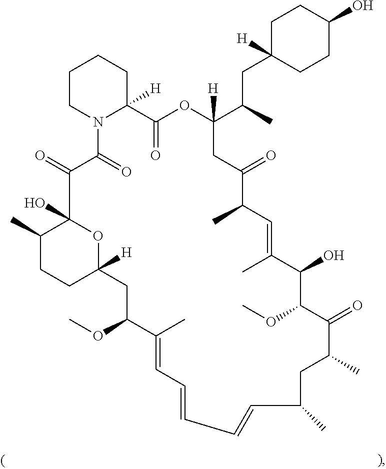

LY294002, LY292223, LY292696, LY293684, LY293646 (Vlahos et al., J. Biol. Chem. 269(7): 5241-5248 (1994)), wortmannin, BAY-43-9006, (Wilhelm et al., Curr. Pharm. Des. 8:2255-2257 (2002)), ZM336372, L-779,450, any Raf inhibitor disclosed in Lowinger et al., Curr. Pharm Des. 8:2269-2278 (2002); flavopiridol (L86-8275/HMR 1275; Senderowicz, Oncogene 19(56): 6600-6606 (2000)) or UCN-01 (7-hydroxy staurosporine; Senderowicz, Oncogene 19(56): 6600-6606 (2000)).

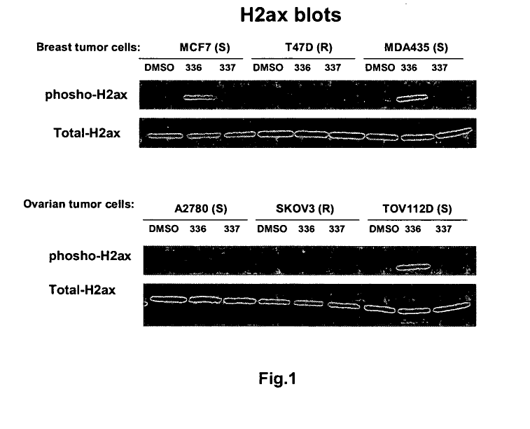

In an embodiment of the invention, an FTI is administered in association with one or more of any of the compounds set forth in U.S. Pat. No. 5,656,655, which discloses styryl substituted heteroaryl EGFR inhibitors; in U.S. Pat. No. 5,646,153 which discloses bis mono and/or bicyclic aryl heteroaryl carbocyclic and heterocarbocyclic EGFR and PDGFR inhibitors; in U.S. Pat. No. 5,679,683 which discloses tricyclic pyrimidine compounds that inhibit the EGFR; in U.S. Pat. No. 5,616,582 which discloses quinazoline derivatives that have receptor tyrosine kinase inhibitory activity; in Fry et al., Science 265 1093-1095 (1994) which discloses a compound having a structure that inhibits EGFR (see FIG. 1 of Fry et al.,); in U.S. Pat. No. 5,196,446 which discloses heteroarylethenediyl or heteroarylethenediylaryl compounds that inhibit EGFR; in Panek, et al., Journal of Pharmacology and Experimental Therapeutics 283: 1433-1444 (1997) which disclose a compound identified as PD166285 that inhibits the EGFR, PDGFR, and FGFR families of receptors-PD166285 is identified as 6-(2,6-dichlorophenyl)-2-(4-(2-diethylaminoethoxy)phenylamino)-8-methyl-8H-pyrido(2,3-d)pyrimidin-7-one.

In an embodiment of the invention, an FTI is administered in association with one or more of any of: pegylated or unpegylated interferon alfa-2a, pegylated or unpegylated interferon alfa-2b, pegylated or unpegylated interferon alfa-2c, pegylated or unpegylated interferon alfa n-1, pegylated or unpegylated interferon alfa n-3 and pegylated, unpegylated consensus interferon or albumin-interferon-alpha.

The term “interferon alpha” as used herein means the family of highly homologous species-specific proteins that inhibit cellular proliferation and modulate immune response. Typical suitable interferon-alphas include, but are not limited to, recombinant interferon alpha-2b, recombinant interferon alpha-2a, recombinant interferon alpha-2c, alpha 2 interferon, interferon alpha-n1 (INS), a purified blend of natural alpha interferons, a consensus alpha interferon such as those described in U.S. Pat. Nos. 4,897,471 and 4,695,623 (especially Examples 7, 8 or 9 thereof), or interferon alpha-n3, a mixture of natural alpha interferons.

Interferon alfa-2a is sold as ROFERON-A® by Hoffmann-La Roche (Nutley, N.J.).

Interferon alfa-2b is sold as INTRON-A® by Schering Corporation (Kenilworth, N.J.). The manufacture of interferon alpha 2b is described, for example, in U.S. Pat. No. 4,530,901.

Interferon alfa-n3 is a mixture of natural interferons sold as ALFERON N INJECTION® by Hemispherx Biopharma, Inc. (Philadelphia, Pa.).

Interferon alfa-n1 (INS) is a mixture of natural interferons sold as WELLFERON® by Glaxo-Smith-Kline (Research Triangle Park, N.C.).

Consensus interferon is sold as INFERGEN® by Intermune, Inc. (Brisbane, Calif.).

Interferon alfa-2c is sold as BEROFOR® by Boehringer Ingelheim Pharmaceutical, Inc. (Ridgefield, Conn.).

A purified blend of natural interferons is sold as SUMIFERON® by Sumitomo; Tokyo, Japan.

The term “pegylated interferon alpha” as used herein means polyethylene glycol modified conjugates of interferon alpha, preferably interferon alpha-2a and alpha-2b. The preferred polyethylene-glycol-interferon alpha-2b conjugate is PEG 12000-interferon alpha-2b. The phrases “12,000 molecular weight polyethylene glycol conjugated interferon alpha” and “PEG 12000-IFN alpha” as used herein include conjugates such as are prepared according to the methods of International Application No. WO 95/13090 and EP1039922 and containing urethane linkages between the interferon alpha-2a or -2b amino groups and polyethylene glycol having an average molecular weight of 12000. The pegylated interferon alpha, PEG 12000-IFN-alpha-2b is available from Schering-Plough Research Institute, Kenilworth, N.J.

The preferred PEG 12000-interferon alpha-2b can be prepared by attaching a PEG polymer to the histidine residue in the interferon alpha-2b molecule. A single PEG 12000 molecule can be conjugated to free amino groups on an IFN alpha-2b molecule via a urethane linkage. This conjugate is characterized by the molecular weight of PEG 12000 attached. The PEG 12000-IFN alpha-2b conjugate can be formulated as a lyophilized powder for injection.

Pegylated interferon alfa-2b is sold as PEG-INTRON® by Schering Corporation (Kenilworth, N.J.).

Pegylated interferon-alfa-2a is sold as PEGASYS® by Hoffmann-La Roche (Nutley, N.J).

Other interferon alpha conjugates can be prepared by coupling an interferon alpha to a water-soluble polymer. A non-limiting list of such polymers includes other polyalkylene oxide homopolymers such as polypropylene glycols, polyoxyethylenated polyols, copolymers thereof and block copolymers thereof. As an alternative to polyalkylene oxide-based polymers, effectively non-antigenic materials such as dextran, polyvinylpyrrolidones, polyacrylamides, polyvinyl alcohols, carbohydrate-based polymers and the like can be used. Such interferon alpha-polymer conjugates are described, for example, in U.S. Pat. No. 4,766,106, U.S. Pat. No. 4,917,888, European Patent Application No. 0 236 987 or 0 593 868 or International Publication No. WO 95/13090.

Pharmaceutical compositions of pegylated interferon alpha suitable for parenteral administration can be formulated with a suitable buffer, e.g., Tris-HCl, acetate or phosphate such as dibasic sodium phosphate/monobasic sodium phosphate buffer, and pharmaceutically acceptable excipients (e.g., sucrose), carriers (e.g. human plasma albumin), toxicity agents (e.g., NaCl), preservatives (e.g., thimerosol, cresol or benzyl alcohol), and surfactants (e.g., tween or polysorbates) in sterile water for injection. The pegylated interferon alpha can be stored as lyophilized powder under refrigeration at 2°-8° C. The reconstituted aqueous solutions are stable when stored between 2° and 8° C. and used within 24 hours of reconstitution. See for example U.S. Pat. Nos. 4,492,537; 5,762,923 and 5,766,582. The reconstituted aqueous solutions may also be stored in prefilled, multi-dose syringes such as those useful for delivery of drugs such as insulin. Typical, suitable syringes include systems comprising a prefilled vial attached to a pen-type syringe such as the NOVOLET® Novo Pen available from Novo Nordisk or the REDIPEN®, available from Schering Corporation, Kenilworth, N.J. Other syringe systems include a pen-type syringe comprising a glass cartridge containing a diluent and lyophilized pegylated interferon alpha powder in a separate compartment.

The scope of the present invention also includes administration of compositions comprising an FTI in association with one or more other anti-cancer chemotherapeutic agents (e.g., as described herein) and optionally (i.e., with or without) in association with one or more antiemetics including, but not limited to, casopitant (GlaxoSmithKline), Netupitant (MGI-Helsinn) and other NK-1 receptor antagonists, palonosetron (sold as Aloxi by MGI Pharma), aprepitant (sold as Emend by Merck and Co.; Rahway, N.J.), diphenhydramine (sold as Benadryl® by Pfizer; New York, N.Y.), hydroxyzine (sold as Atarax® by Pfizer; New York, N.Y.), metoclopramide (sold as Reglan® by AH Robins Co,; Richmond, Va.), lorazepam (sold as Ativan® by Wyeth; Madison, N.J.), alprazolam (sold as Xanax® by Pfizer; New York, N.Y.), haloperidol (sold as Haldol® by Ortho-McNeil; Raritan, N.J.), droperidol (Inapsine®), dronabinol (sold as Marinol® by Solvay Pharmaceuticals, Inc.; Marietta, Ga.), dexamethasone (sold as Decadron® by Merck and Co.; Rahway, N.J.), methylprednisolone (sold as Medrol® by Pfizer; New York, N.Y.), prochlorperazine (sold as Compazine® by Glaxosmithkline; Research Triangle Park, N.C.), granisetron (sold as Kytril® by Hoffmann-La Roche Inc.; Nutley, N.J.), ondansetron (sold as Zofran® by Glaxosmithkline; Research Triangle Park, N.C.), dolasetron (sold as Anzemet® by Sanofi-Aventis; New York, N.Y.), tropisetron (sold as Navoban® by Novartis; East Hanover, N.J.).

Compositions comprising an antiemetic are useful for preventing or treating nausea; a common side effect of anti-cancer chemotherapy. Accordingly, the present invention also includes methods for treating cancer in a subject by administering an FTI optionally in association with one or more other chemotherapeutic agents (e.g., as described herein) and optionally in association with one or more antiemetics.

The present invention further comprises a method for treating or preventing any stage or type of any medical condition set forth herein by administering an FTI in association with a therapeutic procedure such as surgical tumorectomy or anti-cancer radiation treatment; optionally in association with a further chemotherapeutic agent and/or antiemetic, for example, as set forth above.

### Kits and Articles of Manufacture

Kits and articles of manufacture of the present invention include an FTI, e.g., combined with a pharmaceutically acceptable carrier, in a pharmaceutical formulation, e.g., in a pharmaceutical dosage form such as a pill, a powder, an injectable liquid, a tablet, dispersible granules, a capsule, a cachet or a suppository; optionally in association with a further therapeutic agent, e.g., as discussed herein. See for example, Gilman et al. (eds.) (1990), The Pharmacological Bases of Therapeutics, 8^{th} Ed., Pergamon Press; and Remington's Pharmaceutical Sciences, supra, Easton, Pa.; Avis et al. (eds.) (1993) Pharmaceutical Dosage Forms: Parenteral Medications Dekker, New York; Lieberman et al. (eds.) (1990) Pharmaceutical Dosage Forms: Tablets Dekker, New York; and Lieberman et al. (eds.) (1990), Pharmaceutical Dosage Forms: Disperse Systems Dekker, New York.

The kits and articles of manufacture of the present invention may also include information, for example, in the form of a package insert or label indicating that the FTI is intended to be administered to patients or subjects comprising a tumor or malignancy which has been determined to be FTI sensitive by use of a method of making such a determination of FTI sensitivity as discussed herein. The insert or label may take any form, such as paper or on electronic media such as a magnetically recorded medium (e.g., floppy disk) or a CD-ROM.

The label or insert may also include other information concerning the pharmaceutical compositions and dosage forms in the kit or article of manufacture. Generally, such information aids patients and physicians in using the enclosed pharmaceutical compositions and dosage forms effectively and safely. For example, the following information regarding the FTI may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references and patent information.

### Detection of Phospho-Histone H2Ax

The determination of the expression level of a biomarker of the invention (as set forth herein) in a cancerous cell (e.g., in a tumor cell) can be performed using any of the many methods known in the art. In an embodiment of the invention, expression is determined by Western blot, ELISA (enzyme linked immunosorbent assay) or immunohistochemical staining of tissue sections. For example, in an embodiment of the invention, a practitioner evaluates a tumor biopsy sample from a potential subject for FTI therapy for FTI sensitivity using any of the techniques discussed herein. Tumor biopsy techniques are well within the scope of ordinary knowledge of any surgeon (veterinary or human) or clinician.

There are several methods in the art by which a biopsy may be taken from a subject including, for example, endoscopic biopsy, bone marrow biopsy, excisional or incisional biopsy, fine needle aspiration (FNA) biopsy, punch biopsy, shave biopsy, or skin biopsy. Each method is well known to practitioners of ordinary skill in the art and the most appropriate method may be chosen by the practitioner depending on the particulars of the therapeutic situation. Such biopsy techniques are particularly useful, e.g., for testing of solid tumor cells. Blood cancer cells (e.g., leukemic cells in a subject suffering from a leukemia such as CML) may be harvested, in an embodiment of the invention, by drawing blood from the subject, e.g., followed by isolation and testing of the blood cells.

Expression of proteins encoded by biomarkers can also be detected in a tissue of a patient's tumor by western blot analysis. A western blot (also known as an immunoblot) is a method for protein detection in a given sample of cellular or tissue homogenate or extract. It uses gel electrophoresis to separate denatured proteins by mass. The proteins are then transferred out of the gel and onto a membrane (e.g., nitrocellulose or polyvinylidene fluoride (PVDF)), where they are “probed” using antibodies specific to the protein. Anti-phospho histone H2Ax antibodies that recognize a protein in a band on the membrane will bind to it. Following an optional wash, the bound antibodies are then bound by a secondary anti-antibody antibody which is conjugated with a detectable label (e.g., biotin, horseradish peroxidase or alkaline phosphatase) and optionally washed again. Detection of the secondary label signal indicates the presence of the protein. Detection of fluorescence or chemilluminescence may be done by exposure of the membrane to film and developing the film. Such methods are within the capability of a practitioner of ordinary skill in the art.

In an embodiment of the invention, expression of a protein encoded by a biomarker is detected by enzyme-linked immunosorbent assay (ELISA). In an embodiment of the invention, “sandwich ELISA” comprises coating a plate with a capture antibody (e.g., anti-phospho histone H2Ax); adding sample wherein antigen present (e.g., phospho-histone H2Ax) binds to the capture antibody, optionally washing away unbound antigen; adding a detectably linked secondary antibody which also binds to the antigen, optionally washing away unbound secondary antibody; and detecting the secondary antibody. Detection of the signal from the secondary antibody indicates presence of the biomarker antigen protein.

Immunohistochemical staining of tissue sections comprises, in general, visual or optical localization of proteins in a cell or tissue sample, by detection of antibodies which bind to the protein. For example, two strategies used for the immunohistochemical detection of antigens in tissue, are the direct method and the indirect method. In both methods, the tissue is treated to rupture the membranes, e.g., by using detergent such as Triton X-100. In an embodiment of the invention, the direct method comprises reacting a detectably labeled antibody with the antigen in the tissue sections; and detecting the bound antibody.

In an embodiment of the invention, the indirect method comprises reacting an unlabeled primary antibody with the tissue antigen; then reacting a labeled secondary antibody (e.g., a detectably labeled secondary antibody) with the primary antibody; and detecting the presence of the secondary antibody. The secondary antibody can be labeled, e.g., with a fluorescent dye or an enzyme. For example, in an embodiment of the invention, a biotinylated secondary antibody is coupled with streptavidin-horseradish peroxidase. This coupled antibody is reacted with 3,3′-Diaminobenzidine (DAB) to produce a brown staining wherever primary and secondary antibodies are attached in a process known as DAB staining. The reaction can be enhanced using nickel, producing a deep purple/gray staining.

Anti-histone H2Ax antibodies are commercially available from several sources or, alternatively, such antibodies may be raised using commonly known methods. Anti-histone H2Ax antibodies may be used to detect total histone H2Ax and anti-phospho histone H2Ax may be used to detect phosphorylated histone H2Ax. In an embodiment of the invention, the anti-phospho histone H2Ax antibody detects histone H2Ax phosphorylated at serine 139 (i.e., residue 139 is processed histone H2Ax with first methionine removed).

### EXAMPLES

The present invention is intended to exemplify the present invention and not to be a limitation thereof. Any method or composition disclosed herein falls within the scope of the present invention.

### Example 1

### Induction of Phospho-Histone H2Ax Following FTI Treatment Correlates with FTI Sensitivity

In this example, the ability to predict FTI sensitivity in a tumor cell by assaying phospho-histone H2Ax was demonstrated.

### Discussion

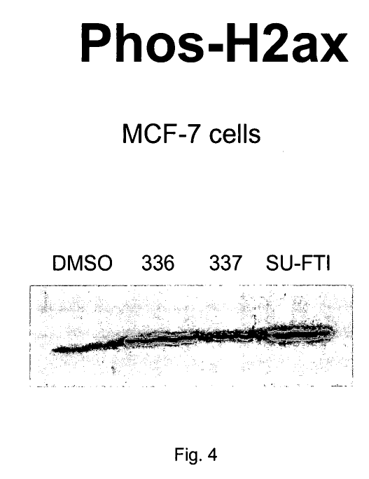

Lonafarnib inhibits cell growth of tumor cell lines to varying degrees. Some cells are especially sensitive (e.g., MCF-7), while others are relatively resistant (e.g., T47D). As is discussed herein, cells which are sensitive to growth inhibition by lonafarnib exhibit increased phosphorylation of histone H2Ax following lonafarnib treatment. This effect is also observed with a structurally-unrelated farnesyl transferase inhibitor (SU-FTI; see FIG. 4). However, treatment of sensitive cell lines with

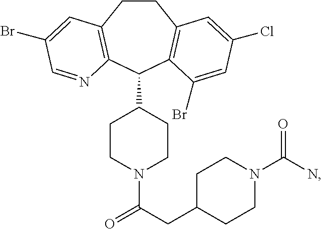

an inactive enantiomer of lonafarnib, which is structurally similar to lonafarnib but fails to inhibit farnesyl transferase, does not induce phosphorylation of histone-H2Ax. Therefore, phosphorylation of histone H2Ax can be used as a marker to predict the response of a cell (sensitivity) to farnesyl transferase inhibitors.

The induction of phospho-histone H2Ax was analyzed by comparing the cellular expression levels of the phosphoprotein following exposure of cells to both DMSO (blank) and lonafarnib. The levels were evaluated by western blot. The quantity of induced phosphoprotein was determined qualitatively and by using densitometric analysis of bands on the blot.

### Results.

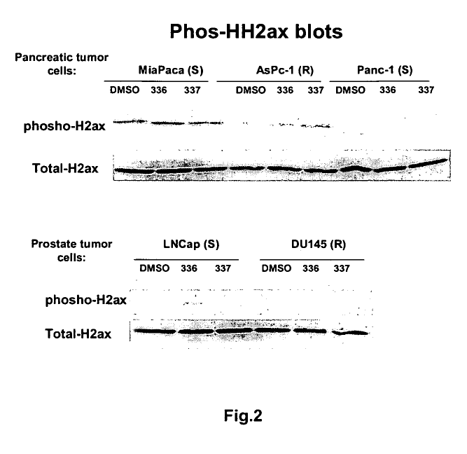

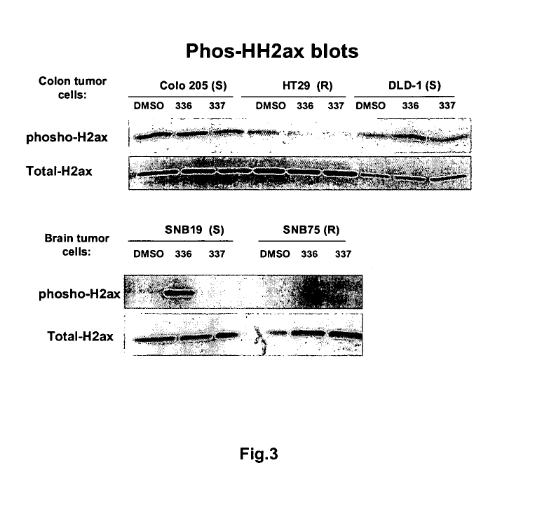

The blots that were generated are set forth in FIGS. 1-4. A set of sensitive cell lines had undetectable basal levels of phospho-histone H2Ax expression. Upon treatment with lonafarnib, phospho-histone H2Ax expression was induced to detectable levels. These cell lines were MCF-7, A2780, SNB19 and TOV112D.

Some lonafarnib sensitive cells lines (MDA-435, MiaPaca, DLD-1 and LNCaP) exhibited low, but detectable basal expression levels of phospho-histone H2Ax which increased significantly upon treatment with lonafarnib. The level of induction of these cells was analyzed densitometrically. The data generated in these experiments is set forth below:
MDA-435: 6.5 fold induction
MiaPaca: 2.3 fold induction
DLD-1: 4.3 fold induction
LNCap: 2.2 fold induction

Lonafarnib resistant cell lines that did not show a significant induction of phospho-histone H2Ax (1-fold difference in OD values) were T47D, SKOV3, SNB75, AsPc1, HT29, DU145. Lonafarnib sensitive cells exhibiting no detectable induction of phos-histone H2Ax upon treatment were Panc1 and Colo205.

Overall, 8 of 10 lonafarnib sensitive cells tested showed induction of phospho-histone H2Ax upon treatment. All resistant lines tested (6 of 6) failed to show an increase in phospho-histone H2Ax upon treatment.

Cells were grown in soft agar and the IC50 for lonafarnib was determined. Cells were designated sensitive (S) or resistant (R). The IC50 value was determined by comparing colony areas of the cell lines exposed to the various doses of FTI. IC50 values were calculated with the XLfit program (idbs; Guildford, United Kingdom).

TABLE 1
Summary of sensitive and resistant cell lines discussed herein.

| Cell Line | Tissue origin | Lonafarnib IC50 (uM) | |
|---|---|---|---|
| T47D | breast | 1.80 | (R) |
| MDA435 | breast | 0.35 | (S) |
| MCF7 | breast | 0.01 | (S) |
| SKOV3 | ovarian | 2.00 | (R) |
| A2780 | ovarian | 0.08 | (S) |
| TOV112D | ovarian | 0.10 | (S) |
| SNB75 | glioma | >3.00 | (R) |
| SNB19 | glioma | 0.23 | (S) |
| MiaPaca | pancreas | 0.24 | (S) |
| Panc1 | pancreas | 0.44 | (S) |
| AsPc1 | pancreas | 1.00 | (R) |
| DLD-1 | colon | 0.32 | (S) |
| Colo205 | colon | 0.33 | (S) |
| HT29 | colon | 0.94 | (R) |
| DU145 | prostate | 1.60 | (R) |
| LNCap | prostate | 0.01 | (S) |

### Methods.

Growth assays. Soft agar assays were performed in 6-well dishes by seeding 10,000-20,000 cells in each well. Cells were plated in top 0.35% low melting point agarose in DMEM:F12 with 10% fetal bovine serum over a bottom 0.6% agarose feeding layer. Cells were grown in the presence of lonafarnib for 14 days and colonies were stained with 1 mg/ml MIT (dimethylthiazol-diphenyl-terrazolium bromide) in PBS. The plates were scanned and the colony area was determined as the sum of the areas stained by MTT.

Western blot protein analysis. Cells were lysed in RIPA buffer (50 mM Tris-HCl, 50 mM NaCl, 1% NP40, 0.5% Na-deoxycholate, 1 mM EDTA, 2.5 mM Na₃VO₄, 20 mM beta-glycerol phosphate, and complete protease inhibitor) and cleared by centrifugation. Protein concentration was determined using BCA reagent. Samples were separated by 14% SDS-PAGE, transferred to polyvinylidene difluoride (PVDF) membrane, immunoblotted and detected by chemiluminescence using the ECL detection reagents. Polyclonal antibodies used: Histone H2Ax and phos-Histone H2Ax (ser-139) (Cell Signaling Technology, Inc.; Danvers, Mass.). Densitometry was analyzed utilizing Quantity One software (Bio-Rad; Hercules, Calif.). Cells that were analyzed for inducation of phospho-H2Ax by western blot analysis were initially plated on plastic and then treated for 72 hours with 1000 nM of the FTI.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Patents, patent applications, publications, product descriptions, and protocols are cited throughout this application, the disclosures of which are incorporated herein by reference in their entireties for all purposes.

I claim:

1. A method for evaluating sensitivity of malignant cells to a farnesyl protein transferase inhibitor comprising determining if said cells exhibit increased expression of phosphorylated histone H2Ax following contact of one or more of said cells with said inhibitor wherein said cells are determined to be sensitive if said increased expression is observed.

2. The method of claim 1 further comprising administering a therapeutically effective dosage of said inhibitor, optionally in association with a further therapeutic agent, to the body of a subject comprising said malignant cells if the cells are determined to be sensitive.

3. The method of claim 1 wherein said farnesyl protein transferase inhibitor is a member selected from the group consisting of lonafarnib; manumycin A; FTI-276; L-744832; BMS-214662; tipifarnib; BMS-316810;

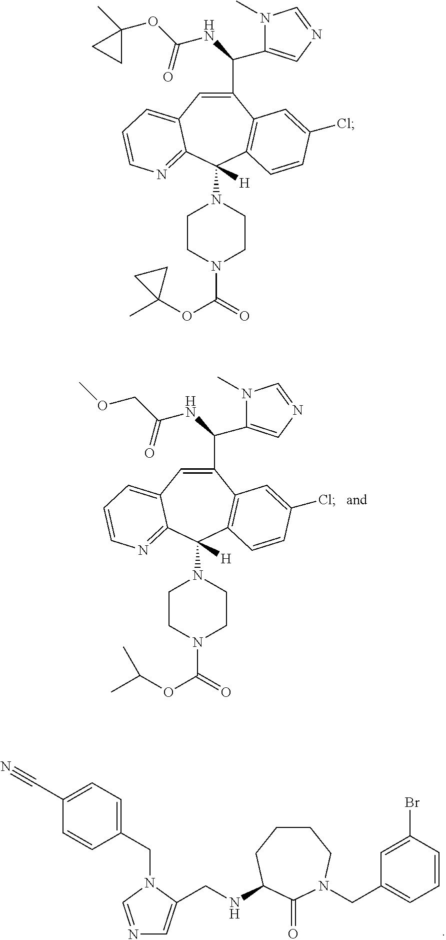

4. The method of claim 1 wherein the malignant cells are in a tumor or mediate a cancerous condition which tumor or condition is selected from the group consisting of a breast tumor, an ovarian tumor, a pancreatic tumor, a prostate tumor, a colon tumor, a brain tumor, a urothelial tract tumor, a non-small cell lung tumor, chronic myelogenous leukemia and a taxane refractory/resistant non-small cell lung tumor.

5. The method of claim 1 comprising:
(a) obtaining a sample of one or more malignant cells from the body of a subject not yet treated with said inhibitor;
(b) determining expression of phosphorylated histone H2Ax in the malignant cells;
(c) treating the subject with the inhibitor;
(d) obtaining a second sample of one or more of said malignant cells from the body of the subject treated with the inhibitor; and
(e) determining expression of the phosphorylated histone H2Ax in the cells obtained from the subject treated with the inhibitor;
wherein the cells are determined to be sensitive to the inhibitor if expression of the phosphorylated histone H2Ax is observed to increase following the treatment with the inhibitor.

6. The method of claim 5 further comprising administering a therapeutically effective dose of said inhibitor, optionally in association with a further therapeutic agent, to said subject, if the cells are determined to be sensitive.

7. The method of claim 5 wherein said farnesyl protein transferase inhibitor is a member selected from the group consisting of lonafarnib; manumycin A; FTI-276; L-744832; BMS-214662; tipifarnib; BMS-316810;

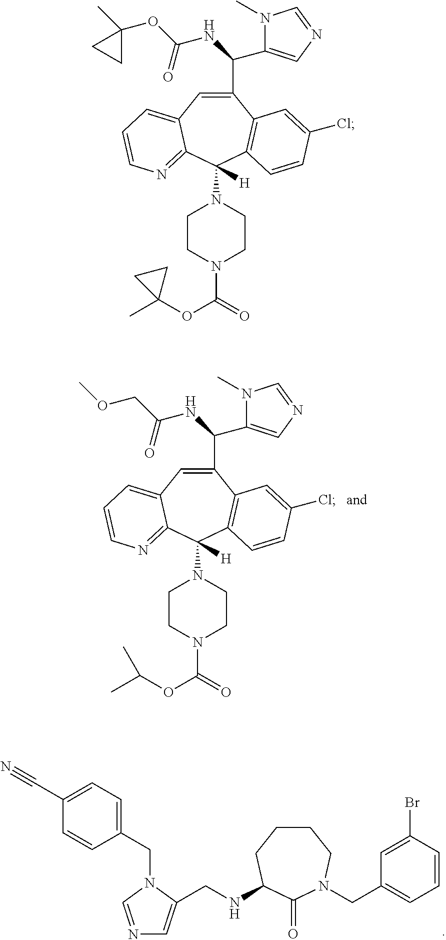

8. The method of claim 5 wherein the malignant cells are in a tumor or mediate a cancerous condition which tumor or condition is selected from the group consisting of a breast tumor, an ovarian tumor, a pancreatic tumor, a prostate tumor, a colon tumor, a brain tumor, a urothelial tract tumor, a non-small cell lung tumor, chronic myelogenous leukemia and a taxane refractory/resistant non-small cell lung tumor.

9. A method for selecting a subject with malignant cells for treatment with a farnesyl protein transferase inhibitor comprising evaluating sensitivity of the malignant cells to said inhibitor by the method of claim 1; wherein said subject is selected if said cells are determined to be sensitive to said inhibitor.

10. The method of claim 9 wherein said farnesyl protein transferase inhibitor is a member selected from the group consisting of lonafarnib; manumycin A; FTI-276; L-744832; BMS-214662; tipifarnib; BMS-316810;

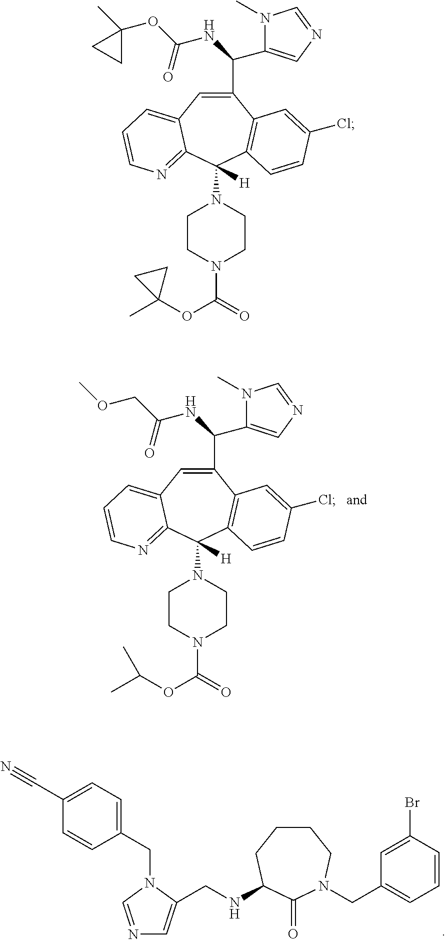

11. The method of claim 9 wherein, after the subject is selected, the subject is administered a therapeutically effective dosage of said farnesyl protein transferase inhibitor optionally in association with a further therapeutic agent.

12. The method of claim 9 wherein the malignant cells are in a tumor or mediate a cancerous condition selected from the group consisting of a breast tumor, an ovarian tumor, a pancreatic tumor, a prostate tumor, a colon tumor, a brain tumor, a urothelial tract tumor, a non-small cell lung tumor, chronic myelogenous leukemia and a taxane refractory/resistant non-small cell lung tumor.

13. A method for identifying a subject with malignant cells sensitive to a farnesyl protein transferase inhibitor comprising evaluating sensitivity of the malignant cells to said inhibitor by the method of claim 1; wherein said subject is identified if said cells are determined to be sensitive to said inhibitor.

14. The method of claim 13 wherein said farnesyl protein transferase inhibitor is a member selected from the group consisting of lonafarnib; manumycin A; FTI-276; L-744832; BMS-214662; tipifarnib; BMS-316810;

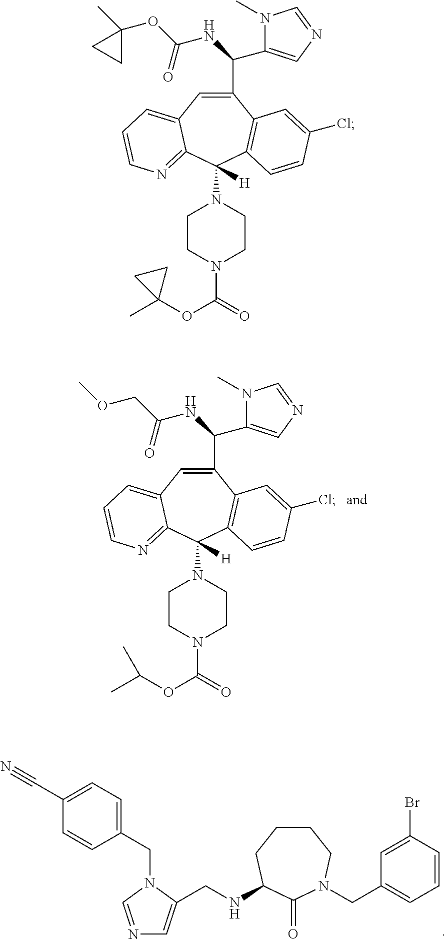

15. The method of claim 13 wherein, after the subject is identified, the subject is administered a therapeutically effective dosage of a farnesyl protein transferase inhibitor optionally in association with a further therapeutic agent.

16. The method of claim 13 wherein the malignant cells are in a tumor or mediate a cancerous condition selected from the group consisting of a breast tumor, an ovarian tumor, a pancreatic tumor, a prostate tumor, a colon tumor, a brain tumor, a urothelial tract tumor, a non-small cell lung tumor, chronic myelogenous leukemia and a taxane refractory/resistant non-small cell lung tumor.

17. A method for treating a tumor or cancerous condition with a farnesyl protein transferase inhibitor comprising evaluating sensitivity of malignant cells, which are in said tumor or which mediate said cancerous condition, to said inhibitor by the method of claim 1 and, if said cells are determined to be sensitive, continuing treatment by administering, to the subject, a therapeutically effective dosage of the inhibitor.

18. The method of claim 17 wherein said farnesyl protein transferase inhibitor is a member selected from the group consisting of lonafarnib; manumycin A; FTI-276; L-744832; BMS-214662; tipifarnib; BMS-316810;

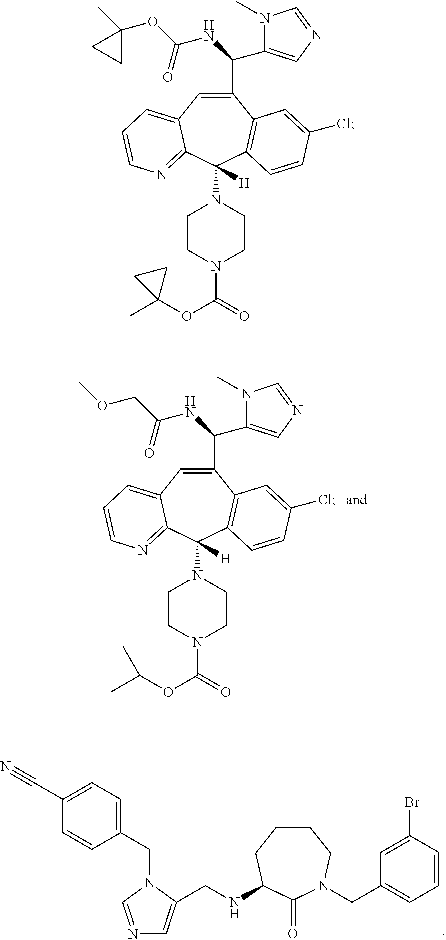

19. The method of claim 17 wherein the farnesyl protein transferase inhibitor is administered in association with a further therapeutic agent.

20. The method of claim 17 wherein the tumor or cancerous condition is selected from the group consisting of a breast tumor, an ovarian tumor, a pancreatic tumor, a prostate tumor, a colon tumor, a brain tumor, a urothelial tract tumor, a non-small cell lung tumor, chronic myelogenous leukemia and a taxane refractory/resistant non-small cell lung tumor.

21. A method for selecting a therapy for a subject with one or more malignant cells comprising evaluating sensitivity of the cells to a farnesyl protein transferase inhibitor by the method of claim 1; wherein said inhibitor is selected as the therapy if said cells are determined to be sensitive to the inhibitor.

22. The method of claim 21 wherein said farnesyl protein transferase inhibitor is a member selected from the group consisting of lonafarnib; manumycin A; FTI-276; L-744832; BMS-214662; tipifarnib; BMS-316810;

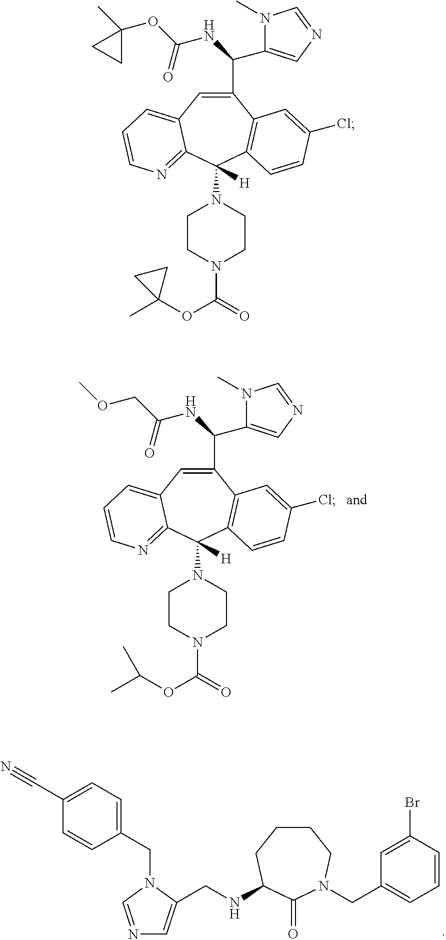

23. The method of claim 21 wherein the malignant cells are in a tumor or mediate a cancerous condition selected from the group consisting of a breast tumor, an ovarian tumor, a pancreatic tumor, a prostate tumor, a colon tumor, a brain tumor, a urothelial tract tumor, a non-small cell lung tumor, chronic myelogenous leukemia and a taxane refractory/resistant non-small cell lung tumor.

24. The method of claim 21 wherein, after the inhibitor is selected as the therapy, the subject is administered a therapeutically effective dosage of the inhibitor optionally in association with a further therapeutic agent.

25. A method of advertising a farnesyl protein transferase inhibitor or a pharmaceutical composition thereof or a therapeutic regimen comprising administration of said inhibitor or composition comprising promoting, to a target audience, the use of the inhibitor or composition for treating a patient or patient population whose tumors or cancerous conditions are mediated by malignant cells that exhibit increased expression of phosphorylated histone H2Ax following an initial treatment with said inhibitor or composition.

26. An article of manufacture comprising, packaged together, a farnesyl protein transferase inhibitor or a pharmaceutical composition thereof comprising a pharmaceutically acceptable carrier; and a label stating that the agent or pharmaceutical composition is indicated for treating patients having a tumor comprising malignant cells or a cancerous condition mediated by malignant cells that exhibit increased expression of phosphorylated histone H2Ax following an initial treatment with said inhibitor.

27. A method for manufacturing a farnesyl protein transferase inhibitor or a pharmaceutical composition thereof comprising a pharmaceutically acceptable carrier said method comprising combining, in a package, the inhibitor or composition; and a label conveying that the inhibitor or composition is indicated for treating patients having a tumor comprising malignant cells or a cancerous condition mediated by malignant cells that express increased levels of phosphorylated histone H2Ax following an initial treatment with said inhibitor.
